(19) United States
(12) Patent Application Publication
(10) Pub. No.: US 2005/0148848 A1
(43) Pub. Date: Jul. 7, 2005

(54) Stereo display of tube-like structures and improved techniques therefor ("stereo display")

(75) Inventors: Yang Guang, Singapore (SG); Eugene Lee Chee Keong, Singapore (SG); Ralf Alfons Kockro, Singapore (SG)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Bracco Imaging, s.p.a., Milano (IT)

(21) Appl. No.: 10/981,058

(22) Filed: Nov. 3, 2004

Related U.S. Application Data
(60) Provisional application No. 60/517,043, filed on Nov. 3, 2003.
(60) Provisional application No. 60/516,998, filed on Nov. 3, 2003.
(60) Provisional application No. 60/562,100, filed on Apr. 14, 2004.

(51) Int. Cl. A61B 5/05

(52) U.S. Cl. 600/407

(57) ABSTRACT

Improved systems and methods for stereoscopically displaying and virtually viewing tube-like anatomical structures are presented. Stereoscopic display of such structures can provide a user with better depth perception of the structure being viewed and thus make a virtual examination more real. In exemplary embodiments according to the present invention, ray shooting, coupled with appropriate error correction techniques, can be utilized for dynamic adjustment of an eye convergence point for stereo display. In exemplary embodiments of the present invention, the correctness of a convergence point can be verified to avoid a distractive and uncomfortable visualization. Additionally, in exemplary embodiments of the present invention, convergence points in consecutive time frames can be compared. If rapid changes are detected, the system can compensate by interpolating transitional convergence points. In exemplary embodiments according to the present invention ray shooting can also be utilized to display occluded areas behind folds and protrusions in the inner colon wall. In exemplary embodiments according to the present invention, interactive display control functionalities can be mapped to a gaming-type joystick or other three-dimensional controller, freeing thereby a user from the limits of a two-dimensional computer interface device such as a standard mouse or trackball.

### CROSS REFERENCE TO OTHER APPLICATIONS

This application claims the benefit of the following United States Provisional Patent applications, the disclosure of each of which is hereby wholly incorporated herein by this reference: Ser. Nos. 60/517,043 and 60/516,998, each filed on Nov. 3, 2003, and Ser. No. 60/562,100, filed on Apr. 14, 2004.

### FIELD OF THE INVENTION

This invention relates to medical imaging, and more precisely to a system and methods for improved visualization and stereographic display of three-dimensional (“3D”) data sets of tube-like anatomical structures.

### BACKGROUND OF THE INVENTION

Historically, the only method by which a health care professional or researcher could view the inside of an anatomical tube-like structure, such as, for example, a blood vessel or a colon, was by insertion of a probe and camera, such as is done in conventional endoscopy/colonoscopy. With the advent of sophisticated imaging technologies such as magnetic resonance imaging (“MRI”) and computerized tomography (“CT”), volumetric data sets representative of luminal (as well as various other) organs can be created. These volumetric data sets can then be rendered to a radiologist or other user, allowing him to inspect the interior of a patient's tube-like organ without having to perform an invasive procedure.

For example, in the area of colonoscopy, volumetric data sets can be created from numerous CT slices of the lower abdomen. In general, from 300-600 or more slices are used in this technique. These CT slices can then be augmented by various interpolation methods to create a three dimensional (“3D”) volume. Portions of the 3D volume, such as the colon, can be segmented and rendered using conventional volume rendering techniques. Using such techniques, a three-dimensional data set comprising a patient's colon can be displayed on an appropriate display. By viewing such a display a user can take a virtual tour of the inside of the patient's colon, dispensing with the need to insert an actual physical instrument. Such a procedure is termed a “virtual colonoscopy.” Virtual colonoscopies (and virtual endoscopies in general) are appealing to patients inasmuch as they involve a considerably less invasive diagnostic technique than that of a physical colonoscopy or other type of endoscopy.

Notwithstanding its convenience and appeal, there are numerous difficulties inherent in a conventional “virtual colonoscopy” or “virtual endoscopy.” Similar problems inhere in the virtual examination of any tube-like anatomical structure using standard techniques. For example, in a conventional “virtual colonoscopy” a user's viewpoint is inside the colon. The viewpoint moves along the colon's interior, usually following a calculated centerline. Conventional virtual colonoscopies are displayed on a standard monoscopic computer display. Thus, environmental depth cues are generally lacking. As a result, important properties of the anatomical structure being viewed go unseen and unnoticed. What is thus needed in the art are improvements to the process of virtual inspections of large tube-like organs (such as a colon or a blood vessel) to optimize the process as well as to take full advantage of the information which is available in a three-dimensional volumetric data set constructed from scan data of the anatomical region containing the tube-like organ of interest. This can best be accomplished via stereoscopic display. Thus, what are needed in the art are improved methods for the real-time stereoscopic display of tube-like structures.

### SUMMARY OF THE INVENTION

Improved systems and methods for stereoscopically displaying and virtually viewing tube-like anatomical structures are presented. Stereoscopic display of such structures can provide a user with better depth perception of the structure being viewed and thus make a virtual examination more real. In exemplary embodiments according to the present invention, ray shooting, coupled with appropriate error correction techniques, can be utilized for dynamic adjustment of an eye convergence point for stereo display. In exemplary embodiments of the present invention, the correctness of a convergence point can be verified to avoid a distractive and uncomfortable visualization. Additionally, in exemplary embodiments of the present invention, convergence points in consecutive time frames can be compared. If rapid changes are detected, the system can compensate by interpolating transitional convergence points. In exemplary embodiments according to the present invention ray shooting can also be utilized to display occluded areas behind folds and protrusions in the inner colon wall. In exemplary embodiments according to the present invention, interactive display control functionalities can be mapped to a gaming-type joystick or other three-dimensional controller, freeing thereby a user from the limits of a two-dimensional computer interface device such as a standard mouse or trackball.

Further features of the invention, its nature and various advantages will be more apparent from the accompanying drawings and the following detailed description of the various exemplary embodiments.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIGS. 1A and 1B respectively depict a conventional monoscopic rendering of a “cave” and a polyp from an exemplary colon segment;

FIGS. 1(a)A and 1(a)B are grayscale versions of FIGS. 1, respectively;

FIGS. 2 depict a stereoscopic rendering of the polyp of FIG. 1B according to an exemplary embodiment of the present invention;

FIGS. 2(a) are grayscale versions of FIGS. 1, respectively;

FIG. 3 depicts an exemplary polyp in an exemplary colon segment rendered in anaglyphic red-green stereo according to an exemplary embodiment of the present invention;

FIG. 3(a) is a grayscale version of the Left or red channel of FIG. 3;

FIG. 3(b) is a grayscale version of the Right or green channel of FIG. 3;

FIG. 3A depicts an exemplary colon segment rendered stereoscopically according to an exemplary embodiment of the present invention;

FIG. 3A(a) is a grayscale version of the Left or red channel of FIG. 3A;

FIG. 3A(b) is a grayscale version of the Right or green channel of FIG. 3A;

FIG. 3B is the exemplary colon segment of FIG. 3A with certain areas denoted by index numbers;

FIG. 3B(a) is a grayscale version of the Left or red channel of FIG. 3B;

FIG. 3B(b) is a grayscale version of the Right or green channel of FIG. 3B;

FIG. 3C is a monoscopic view of an exemplary magnified portion of the colon segment of

FIGS. 3A and 3B according to an exemplary embodiment of the present invention;

FIGS. 3D and 3E, are red-blue and red-cyan, respectively, anaglyphic stereoscopic renderings of the exemplary magnified colon segment of FIG. 3C according to exemplary embodiments of the present invention;

FIGS. 3D and 3E, are red-blue and red-cyan, respectively, anaglyphic stereoscopic renderings of the exemplary magnified colon segment of FIG. 3C according to exemplary embodiments of the present invention;

FIG. 3F is a red-green anaglyphic stereoscopic rendering of the exemplary magnified colon segment of FIG. 3C according to an exemplary embodiment of the present invention;

FIG. 3F(a) is a grayscale version of the Left or red channel of FIG. 3F;

FIG. 3F(b) is a grayscale version of the Right or green channel of FIG. 3F;

FIG. 3G is a monoscopic display of two diverticula of an exemplary colon segment according to an exemplary embodiment of the present invention;

FIGS. 3H, 3I and 3J are red-blue, red-cyan and red-green, respectively, anaglyphic stereoscopic renderings of the exemplary colon segment depicted in FIG. 3G according to exemplary embodiments of the present invention;

FIG. 3J(a) is a grayscale version of the Left or red channel of FIG. 3J;

FIG. 3J(b) is a grayscale version of the Right or green channel of FIG. 3J;

FIG. 4 depicts a conventional overall image of an exemplary tube-like structure;

FIG. 4(a) is a grayscale version of FIG. 4;

FIG. 5 depicts an exemplary overall image of a colon in red-green stereo according to an exemplary embodiment of the present invention;

FIG. 5(a) is a grayscale version of the Left or red channel of FIG. 5;

FIG. 5(b) is a grayscale version of the Right or green channel of FIG. 5;

FIGS. 6(a)-(c) illustrate calculating a set of center points through a tube-like structure by shooting out rays according to an exemplary embodiment of the present invention;

FIG. 6A depicts an exemplary ray shot form point A to point B in a model space, encountering various voxels on its way;

FIGS. 7(a)-(f) illustrate the ray shooting of FIGS. 6 in greater detail according to an exemplary embodiment of the present invention;

FIGS. 8(a)-(d) illustrate correction of an average point obtained by ray shooting according to an exemplary embodiment of the present invention;

FIG. 9 illustrates shooting rays to verify the position of an average point according to an exemplary embodiment of the present invention;

FIG. 10 is a top view of two eyes looking at two objects while focusing on a given example point;

FIG. 11 is a top view of two cameras focused on the same point;

FIG. 12 is a perspective side view of the cameras of FIG. 11;

FIGS. 13 illustrate the left and right views, respectively, of the cameras of FIGS. 11 and 12;

FIG. 14 depicts the placement of a viewer's position, eye position and direction according to an exemplary embodiment of the present invention;

FIGS. 15(a)-(c) illustrate correct, incorrect—too near, and incorrect—too far convergence points, respectively, for two exemplary cameras viewing an example wall;

FIG. 16 illustrates a top view of two eyes looking at two objects;

FIG. 17(a) illustrates an exemplary image of the two objects of FIG. 16 as seen by the left eye;

FIG. 17(b) illustrates an exemplary image of the two objects of FIG. 16 as seen by the right eye;

FIG. 18(a) illustrates a correct convergence at point A for viewing a region according to an exemplary embodiment of the present invention;

FIG. 18(b) illustrates an incorrect convergence at point B for viewing the region which is too far away;

FIG. 128(c)illustrates a incorrect convergence at point C for viewing the region which is too near;

FIG. 19 illustrates determining convergence points according to an exemplary embodiment of the present invention;

FIG. 20 depicts the situation where an obstruction in one eye's view occurs;

FIG. 21 illustrates slowing down the change of the convergence point with respect to position according to an exemplary embodiment of the present invention;

FIG. 22 depicts a fold in an exemplary colon wall and a “blind spot” behind it, detected according to an exemplary embodiment of the present invention;

FIG. 23 depicts an exemplary joystick with various control interfaces; and

FIG. 24 depicts an exemplary stylus and an exemplary six-degree of freedom controller used to interactively control a display according to an exemplary embodiment of the present invention.

It is noted that the patent or application file contains at least one drawing executed in color. Copies of this patent or patent application publication with color drawings will be provided by the U.S. Patent Office upon request and payment of the necessary fee.

Because numerous grayscale versions of various color drawings are presented herein it is understood that any reference to a color drawing is also a reference to its counterpart grayscale drawing, and vice versa. For economy of presentation, a description of or reference to a given color drawing will not be repeated vis-à-vis its grayscale counterpart, it being understood that the description equally applies to such counterpart unless specifically noted otherwise.

### DETAILED DESCRIPTION OF THE INVENTION

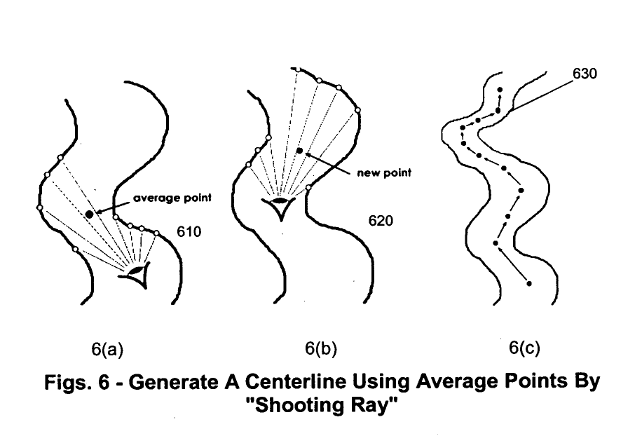

In exemplary embodiments of the present invention a ray can be constructed starting at any position in the 3D model space and ending at any other position in the 3D model space. By checking the values of each voxel that such a ray passes through relative to a defined threshold value, such an exemplary system can obtain information regarding the “visibility” of any two points. For example, as depicted in FIG. 6A, a ray can be constructed that originates at point A and terminates at point B. On its path it passes through a number of voxels. If none of those voxels has an intensity value that is larger than the given threshold value, then those voxels through which it passes are “invisible” and points A and B are visible to each other. On the other hand, if in the path taken by the ray the intensity value of a given voxel becomes larger than the threshold value, than points A and B are said to be blocked by that voxel, and are invisible to each other. Thus, the first point where the ray hits an obstructing voxel, for example point C in FIG. 6A, is the maximum visibility distance from point A along the direction from point A to point B. This distance, i.e. the distance between points A and C, can be calculated. Techniques involving shooting rays, inter alia, are utilized in exemplary embodiments of the present invention to improve upon the interactive display of three-dimensional tube-like structures.

Stereo Display

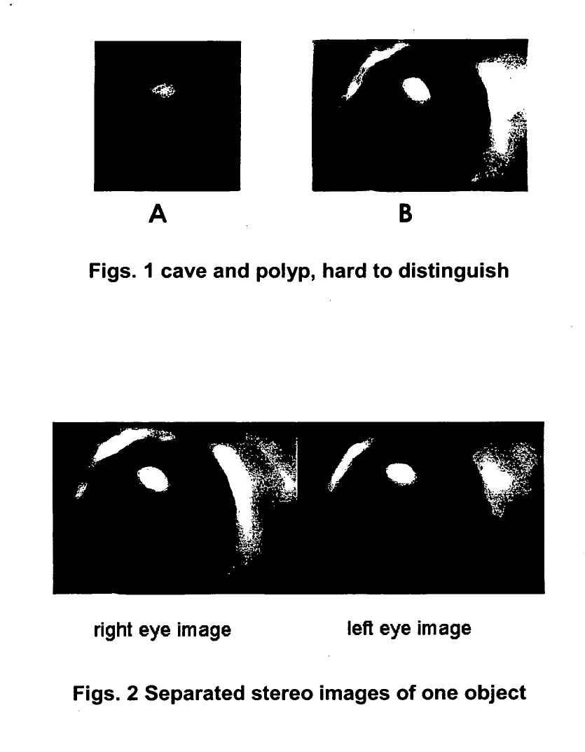

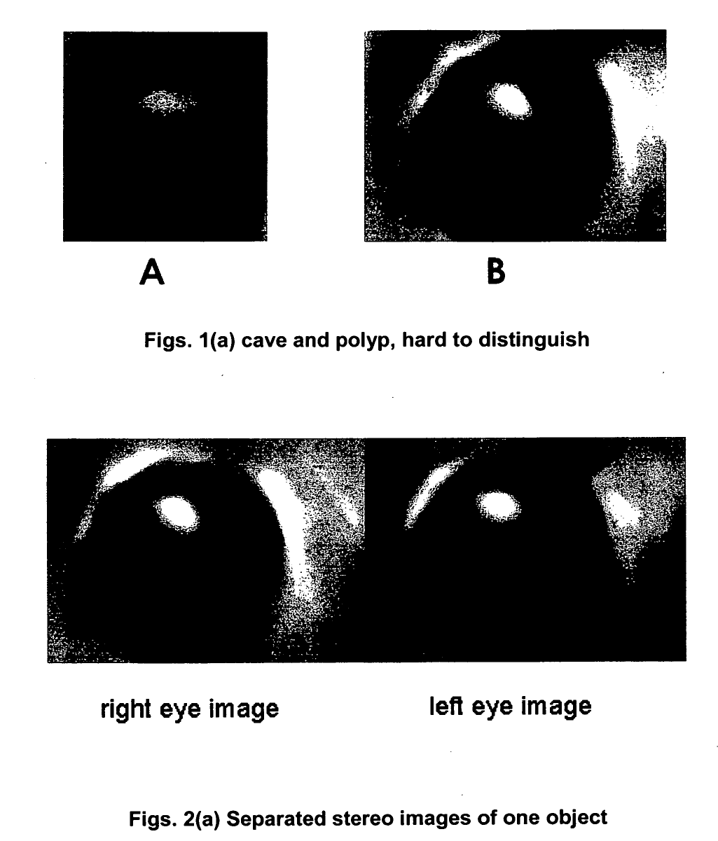

In exemplary embodiments according to the present invention, a tube-like anatomical structure can be displayed stereoscopically so that a user can gain a better perception of depth and can thus process depth cues available in the virtual display data. If presented monoscopically, an interior view of a lumen wall from a viewpoint within the lumen can make it difficult to distinguish an object on the lumen wall which “pops up” towards a user from a concave region or hole in the wall surface which “retreats” from the user. Illustrating this situation, FIG. 1A depicts an exemplary concave region or “cave” and FIG. 1B an exemplary polyp, which is convex to someone whose viewpoint is within the colon lumen. These structures are difficult to distinguish when displayed monoscopically.

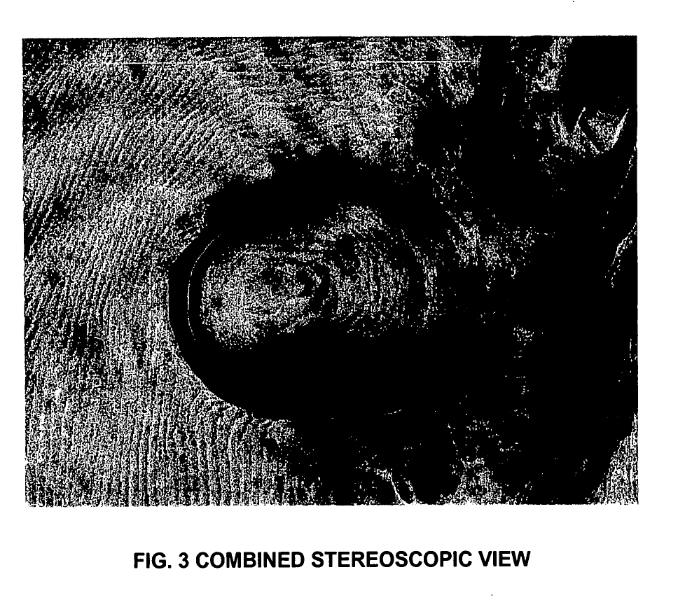

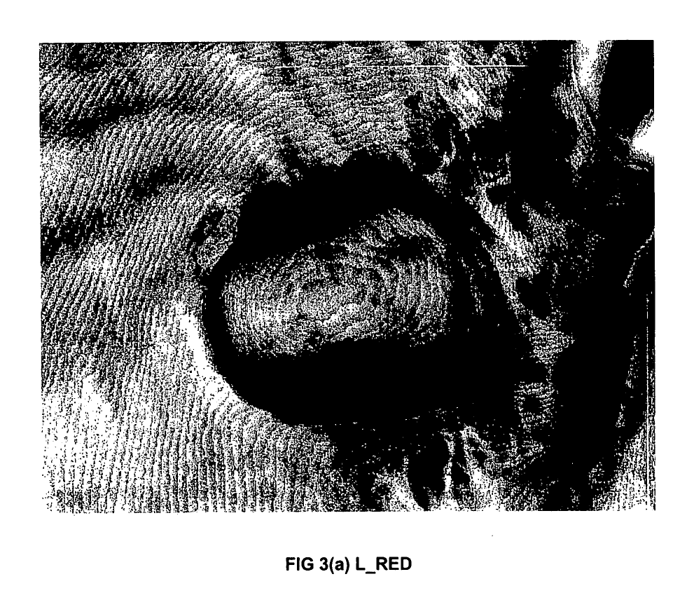

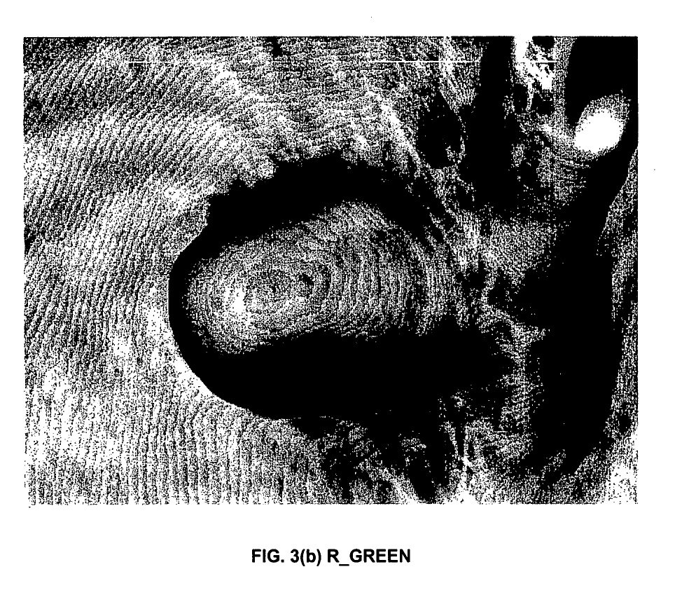

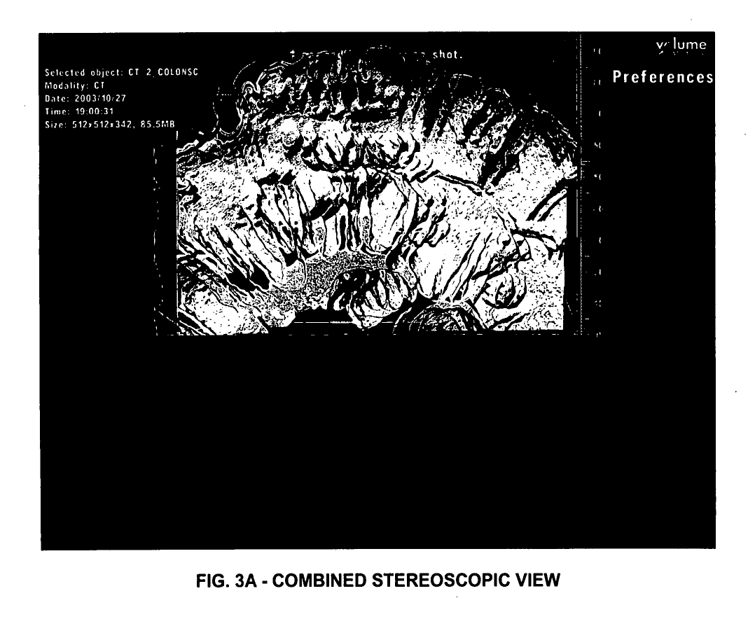

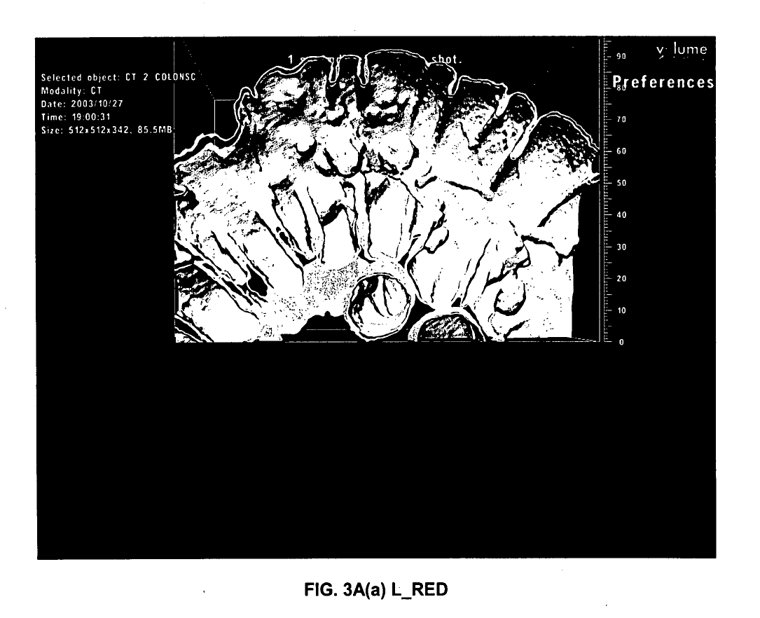

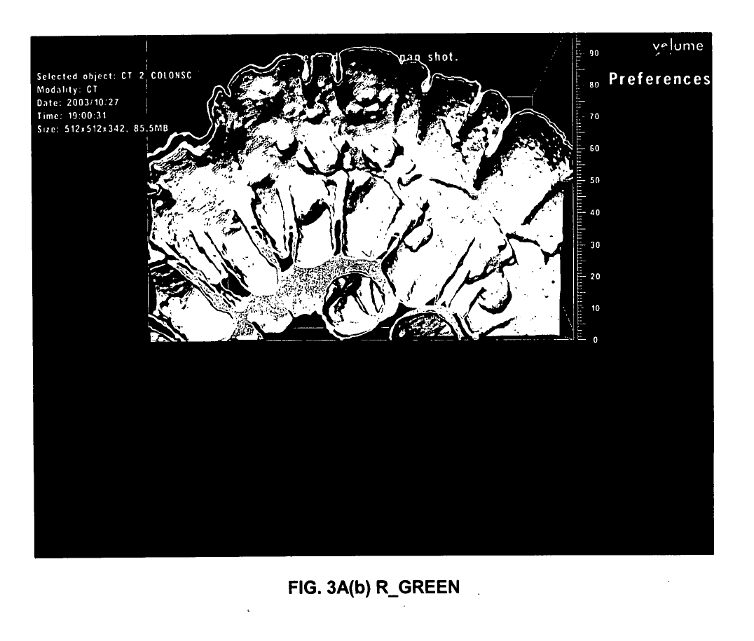

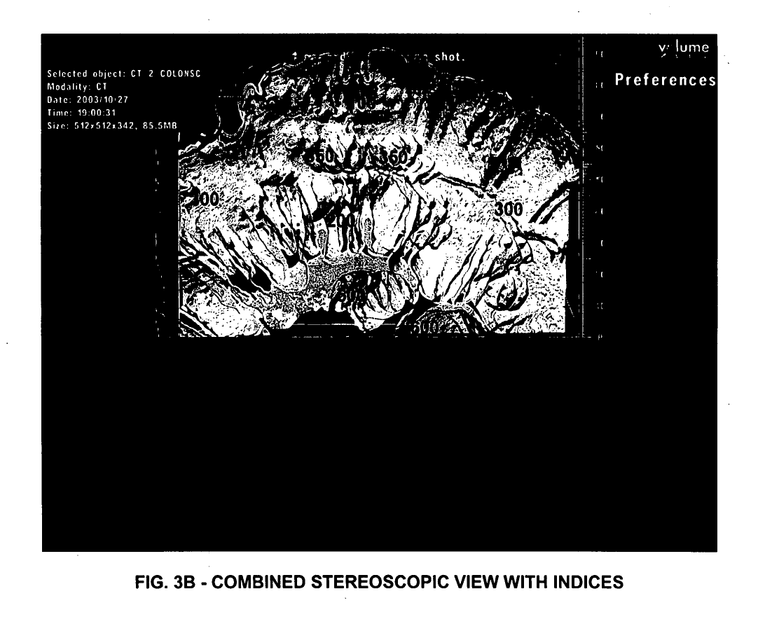

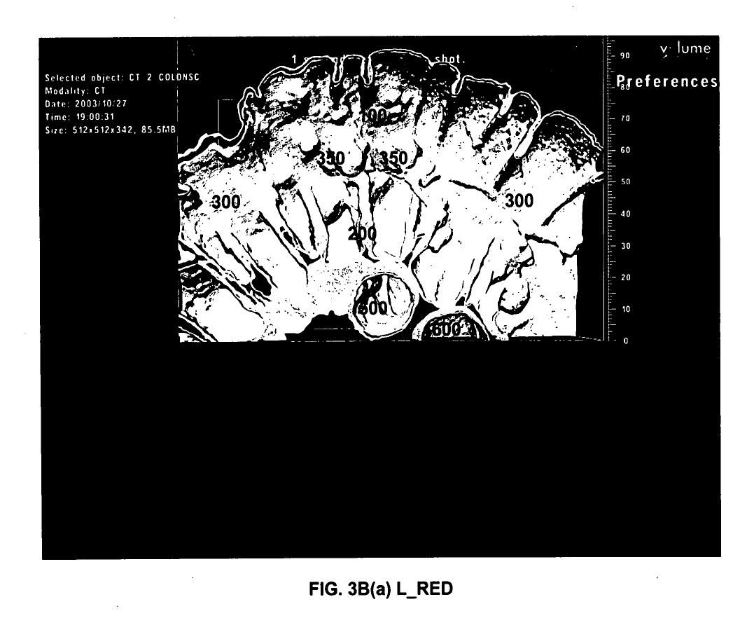

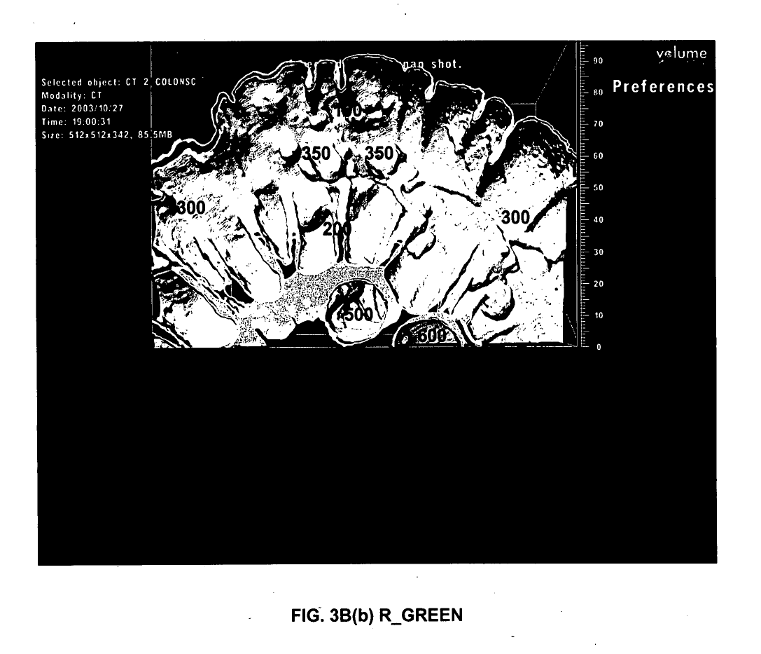

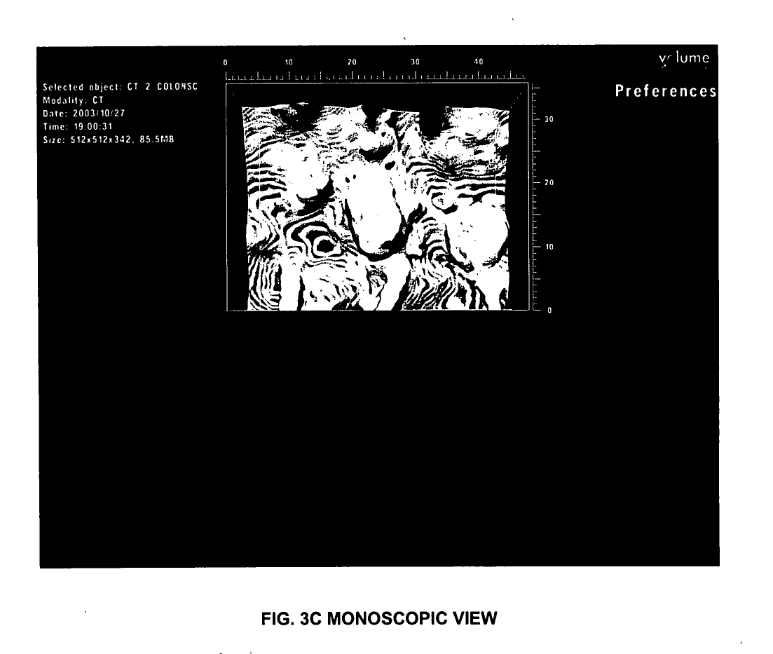

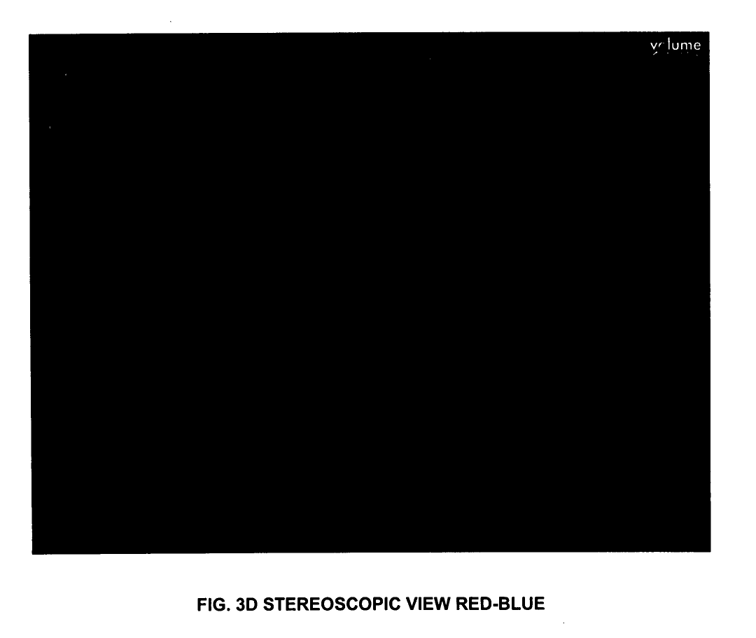

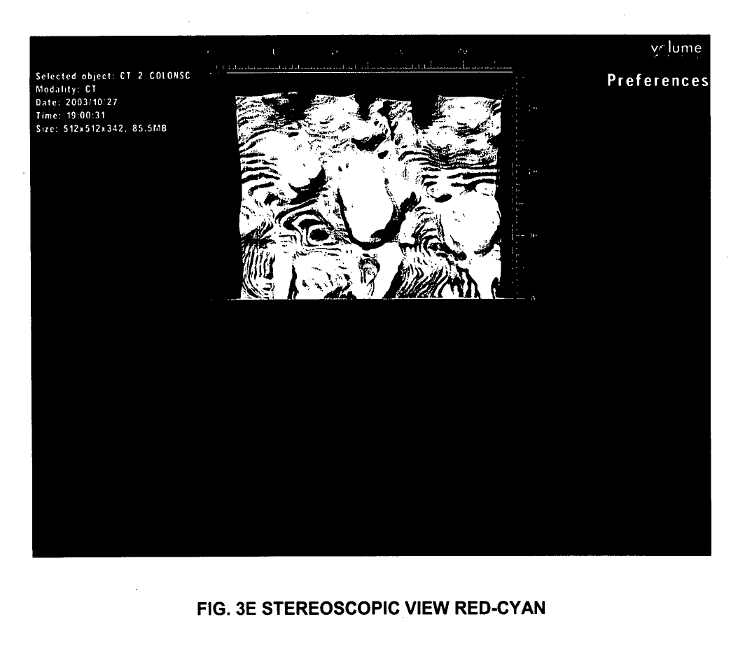

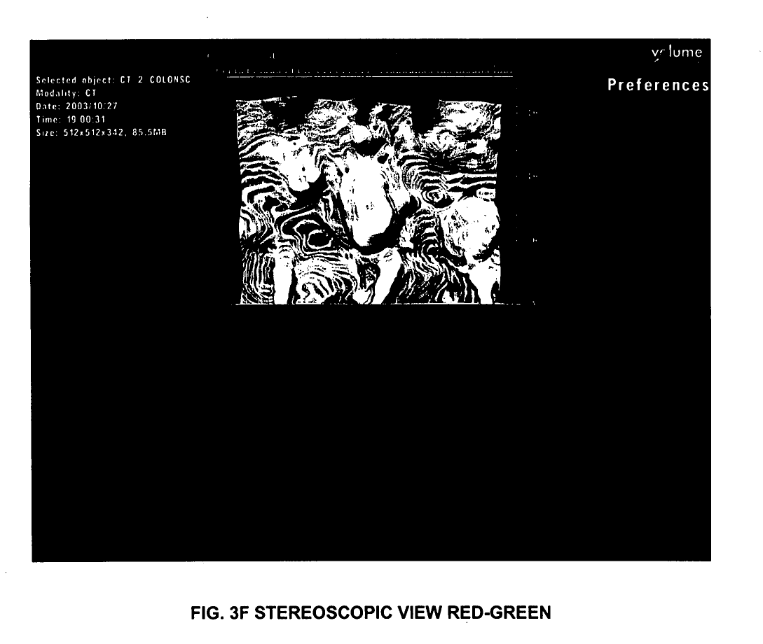

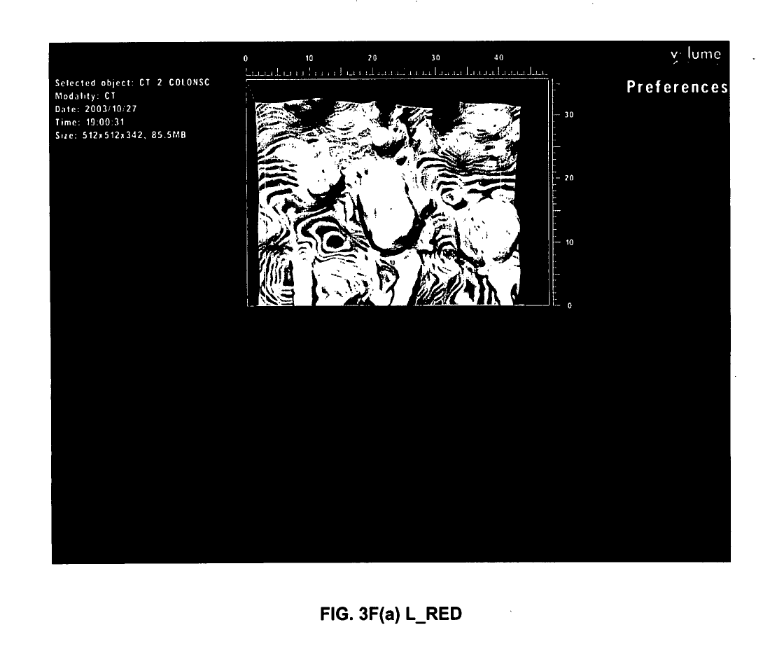

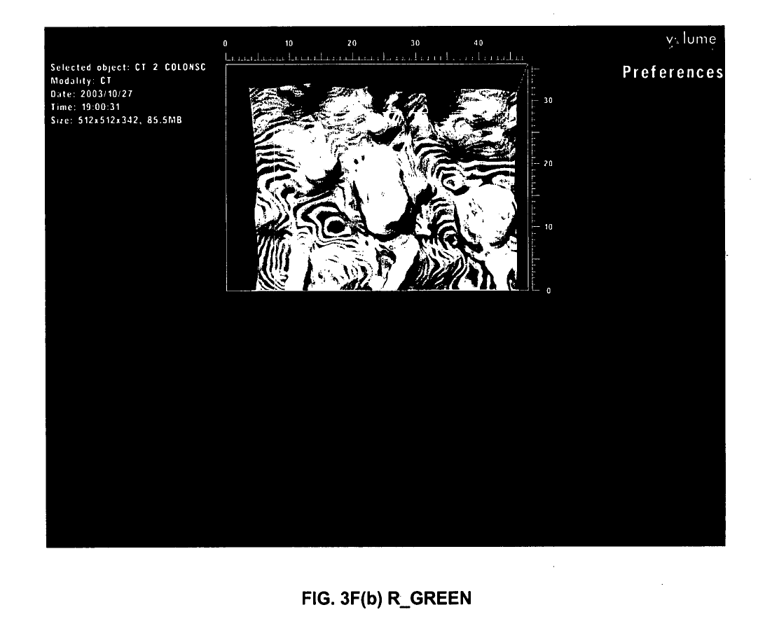

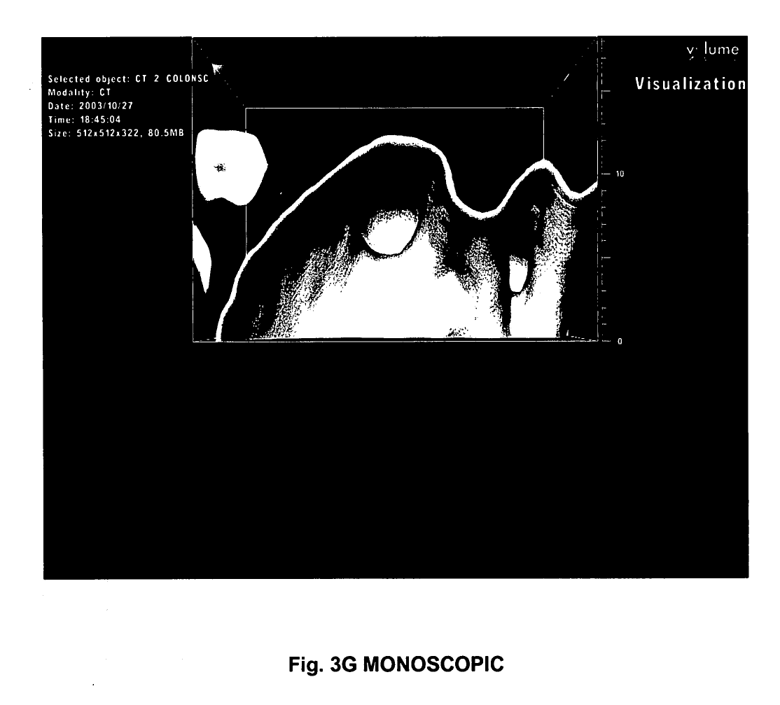

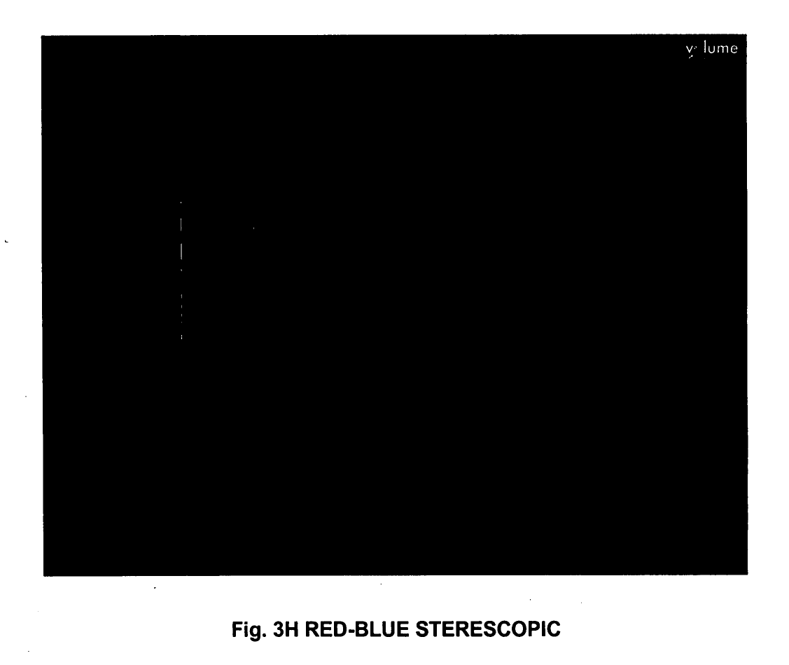

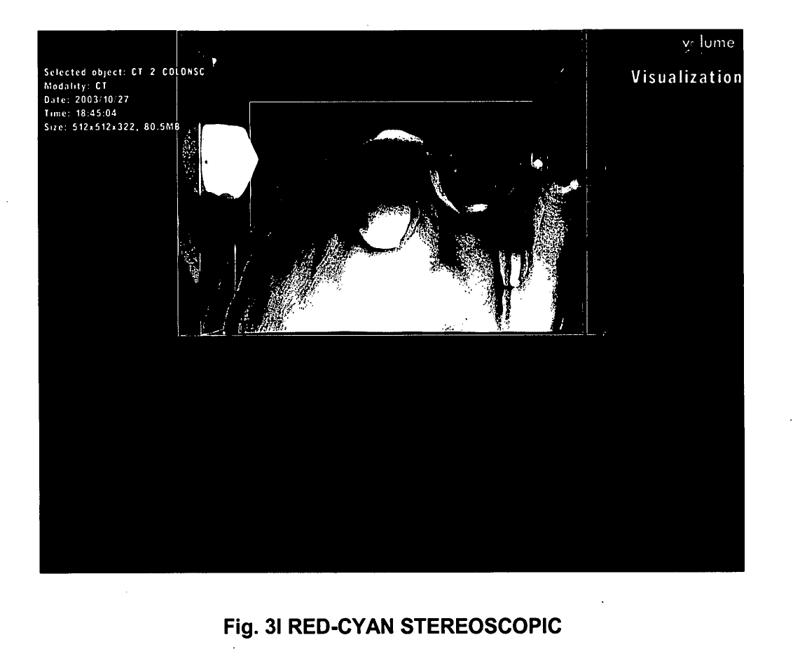

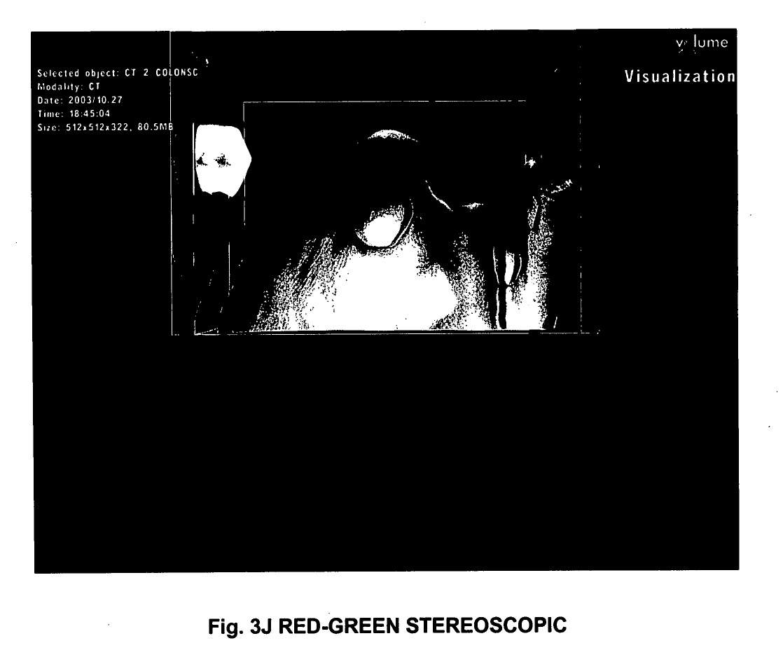

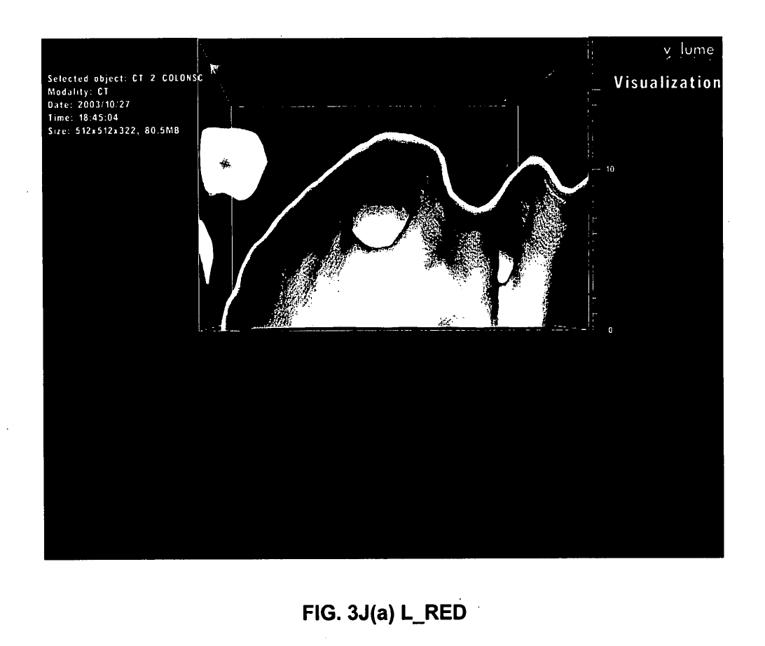

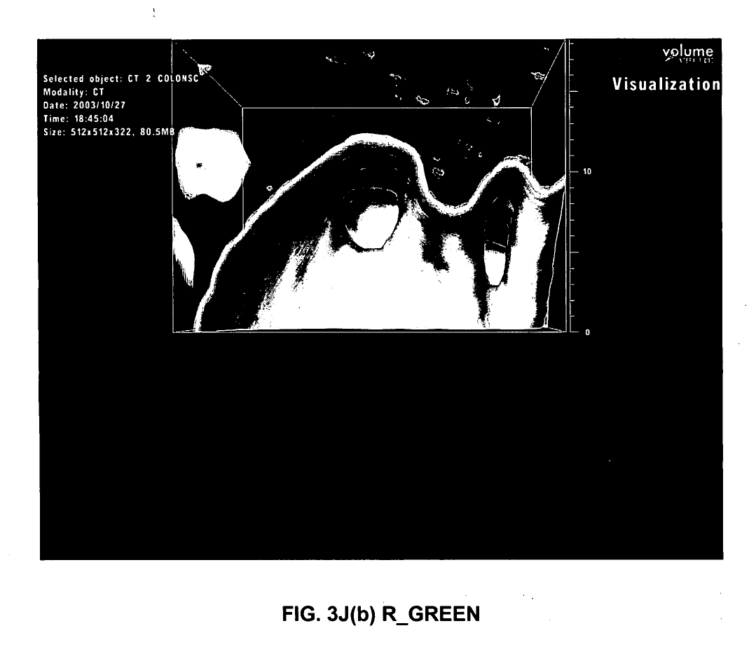

Presenting a virtual display in stereo can resolve this ambiguity. For example, FIG. 2 illustrates images of an object (the polyp of FIG. 1B) generated for left and right eyes, respectively. With, for example, an interlaced display and 3D viewing glasses, a user can easily tell from a stereo display of this object that it is a polyp “popping up” from its surroundings. The stereo effect of the combined images of FIG. 2 can be viewed by crossing the eyes, and having the left eye look at the “left eye” image on the right of the figure and the right eye look at the “right eye” image on the left of the figure. FIG. 3 shows another exemplary object from a colon wall in anaglyphic red-green stereo (to be viewed with red-green glasses, commonly available in magic and scientific project shops). The object is a polyp protruding from the colon wall. In an analogous fashion to FIG. 2, FIGS. 3(a) and 3(b) depict the Left (red) and Right (green) channels of FIG. 3, respectively. By holding FIGS. 3(a) and 3(b) side by side (i.e., L on the right, R on the left) and crossing one's eyes, the stereo effect can also be seen. This manner of viewing images stereoscopically applies to each of the component Left and Right channel pairs of each stereoscopic image presented herein. For economy of description it shall be understood as implicit and not reiterated each time a component Left and Right channel pair of images are described or discussed.

FIGS. 3A through 3J further depict the advantages of stereoscopic display in the examinations of tube-like anatomical structures such as, for example, a human colon. With reference to FIG. 3A, there is depicted stereoscopically an exemplary colon segment. The exemplary colon segment is rendered using anaglyphic red-green stereo. Viewed with proper glasses, which can be as simple as the red-green “3D viewing glasses” available in many magic stores, educational/scientific stores, and even toy stores, one can immediately appreciate the sense of depth perception that can only be gained using stereoscopic display. In FIG. 3A the folds of the colon along the upper curve of the colon are rendered with all of their depth cues and three-dimensional information readily visible. FIGS. 3A(a) and (b) respectively depict the L and R channels of the stereoscopic image shown in FIG. 3A.

FIG. 3B depicts the exemplary colon section of FIG. 3A with certain sections of the image marked with index numbers so that they can be better described. FIGS. 3B(a) and (b) respectively depict the L (red) and R (green) channels of the stereoscopic image shown in FIG. 3B. With reference to FIG. 3B, there are visible upper folds 100, as well as lower folds 200 of the upper colon segment 300. In FIG. 3B the upper colon segment, which is essentially bisected longitudinally by the forward plane of the zoom box (perceived as the forward vertical plane of the display device) is visible, as are two lower colon segments 500 and 600, apparently not connected to the upper colon segment. Below upper colon segment 300, which occupies most of FIG. 3B, at the bottom center of the figure are visible the two other colon segments 500 and 600. These are bisected axially by the forward plane of the zoom box such that one can look through them in more or less endoscopic view. Between the upper folds 100 and the lower folds 200 of the upper colon segment are visible two protrusions 350 which appear to be polyps. A rectangular area surrounding these two potential polyps is what is presented in FIGS. 3C through 3F at higher magnification.

With reference to FIG. 3C, one can see the two polyps (350 with reference to FIG. 3B), and their surrounding tissues. One polyp appears at the center of the image, and the other at the right edge of the image. Because FIG. 3C is a monoscopic rendering of this area certain depth information is not readily available. It is not easy to ascertain the direction and amount of protrusion of these suspected polyps relative to the surrounding area of the inner lumen wall.

FIGS. 3D through 3F are anaglyphic stereoscopic renderings of the magnified exemplary colon segment presented in FIG. 3C. FIG. 3D depicts the image in red-blue stereo, FIG. 3E in red-cyan stereo, and FIG. 3F in red-green stereo. As can be seen from viewing FIGS. 3D through 3F with proper stereoscopic glasses, the available depth cues are readily apparent and one can see the protrusions of the suspected polyp areas, their directions of protrusion form the inner lumen wall, and the contouring of their surrounding tissues. FIGS. 3F(a) and (b) respectively depict the L and R channels of the stereoscopic image shown in FIG. 3F. The L (red) and R (green) channels of each of FIGS. 3D and 3E are essentially identical to FIGS. 3F(a) and (b).

FIGS. 3G through 3J depict another exemplary colon segment, which contains concave “holes” or diverticula, as next described. With reference to FIG. 3G, one can see two diverticula, one at the center and one near the far right of the image, visible in the depicted colon segment. Because FIG. 3G is depicted monoscopically, although one can see the shapes of the suspected diverticula it is not immediately clear whether or not they are concave regions relative to their surrounding tissue, or are convex regions. This ambiguity is resolved when viewing the same image stereoscopically, as displayed in exemplary embodiments of the present invention as is depicted, for example, in FIGS. 3H, 3I, and 3J. With reference to FIGS. 3H, 3I, and 3J, which are rendered using different stereo formats (i.e., red-blue, red-cyan and red-green stereo, respectively), one can immediately appreciate the depth information and perceive that the two suspected regions are, in fact, concave with reference to their surrounding tissue. Thus, one can tell that these regions are in fact diverticula or concave “hole” regions of the depicted example colon.

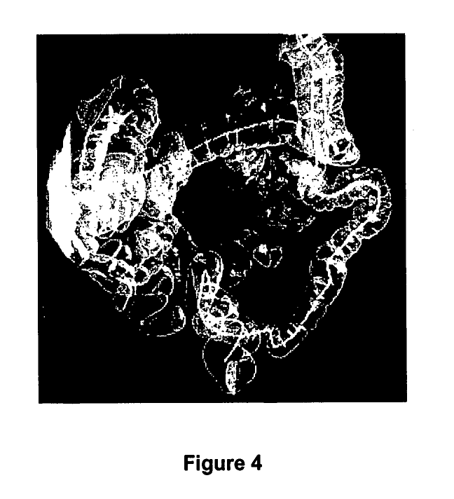

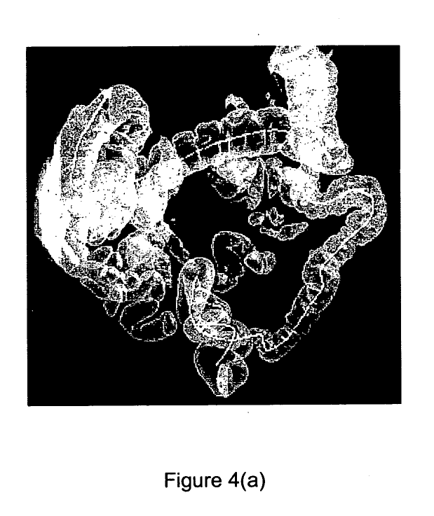

Additionally, in exemplary embodiments according to the present invention, stereoscopic display techniques can also be used for an overall “map” image of a structure of interest. For example, FIG. 4 depicts a conventional “overall map” popular in many virtual colonoscopy display systems, and FIG. 4(a) presents a grayscale version. As can be seen with reference to FIG. 4, such a map can give a user position and orientation information as he travels up or down a tube-like organ such as, for example, the colon. Such a map can, for example, in exemplary embodiments of the present invention, be displayed alongside a main viewing window (which can, for example, provide a localized view of a portion of the tube-like structure), and a user can thereby track his overall position within the tube-like structure as he moves within it in the main viewing window.

With the display of additional visual aids, such an overall view map can, besides indicating the user's current position and orientation, also display the path a user has passed during the navigation. Notwithstanding the usefulness of such a map, displaying it monoscopically cannot give a user much, if any, depth information. Depth information can be very important when parts of the displayed structure appear to overlap, as is often the case when displaying a colon. For example, with reference to FIGS. 4, the respective upper-left and upper-right parts of the displayed colon show that in these areas the displayed colon overlaps itself. However, without depth cues a viewer cannot tell which portion is on top (or forward in the display relative to a user viewpoint) and which is underneath (or backward in the display relative to a user viewpoint). To resolve such ambiguities, in exemplary embodiments according to the present invention, a stereoscopic image of the overall structure or “map” view can be displayed stereoscopically with additional visual aids (such as, for example, a curve to indicate the path traversed thus far and/or an arrow to indicate the current position and viewing direction). Such a display can provide a user with clearer and more intuitive depth and orientative information.

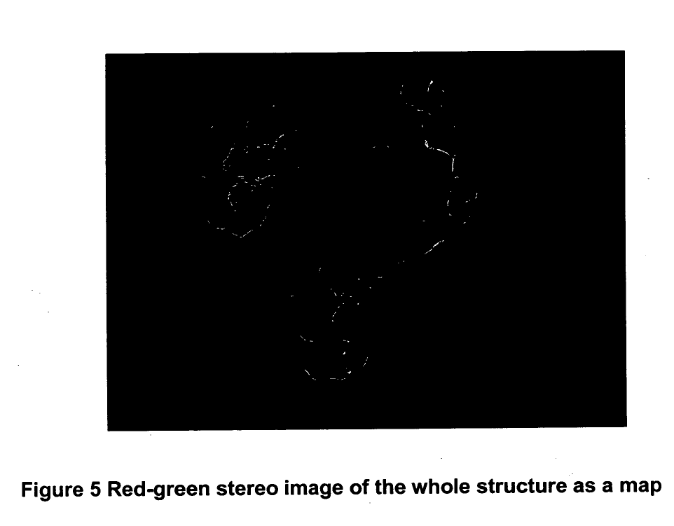

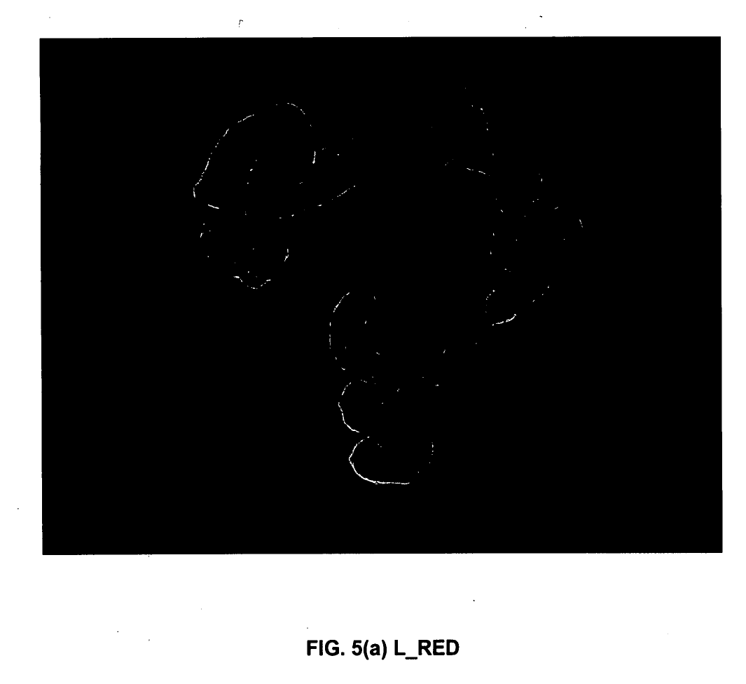

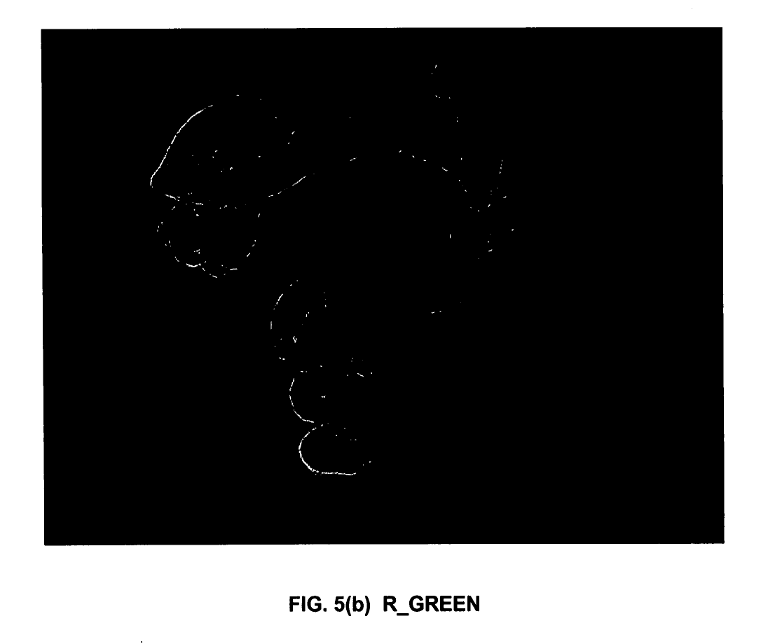

Thus, an example of a stereoscopically rendered overall view according to an exemplary embodiment of the present invention is depicted in FIG. 5. In the example shown in FIG. 5, two slightly different static images of the whole colon were pre- rendered for left eye and right eye viewing angles, respectively. These images can, for example, be used to display a stereo image during run time where only the position and pathway traversed are updated, instead of re-rendering the stereo image in every display loop. This can, for example, save computing resources with no resulting loss of information inasmuch as the depicted view of the entire colon is essentially fixed, being a map view. Thus the shape of the structure does not change during the process. The elements of the map view that do change, i.e., the visual aids, are stereoscopically displayed dynamically but with very low rendering cost. In alternate exemplary embodiments according to the present invention, where the shape, orientation and position of a colon or other tube-like structure as a whole may, for example, change relative to position (scan or viewing) along the colon lumen, the entire colon can be continually stereoscopically re-rendered in the map window as a user moves through it. FIGS. 5(a) and 5(b) are grayscale versions of the Left (Red) and Right (Green), respectively, channels of FIG. 5.

Optimized Center Line Generation

In exemplary embodiments according to the present invention, a ray-shooting algorithm as described above can be used in various ways to optimize the interactive display of a tube-like structure. For example, inside an exemplary tube-like structure, at any starting position, a series of rays can, for example, be emitted into the 3D space, as shown in FIG. 6(a). The rays will ultimately collide with the inner walls of the structure, and the coordinates of the resultant “hit points” (points on the surface of the wall that are hit by the emitted rays) can be calculated and recorded.

If a sufficient number of rays are shot, the resultant “hit points” (i.e., the white dots on the surface of the lumen in FIG. 6(a)) can actually roughly describe the shape of the interior space of the tube-like structure. For example, if the structure were a cylinder, then all the hit points would be on the surface of such cylinder, and thus all the hit points together would form the shape of a cylinder.

Using the 3D coordinates of the set of hit points, an average point 610 can be calculated by averaging the coordinates of all of the hit points. Since it is an average, this point will fall approximately at the center of the portion of the structure that is explored by the rays.

The resultant average point can then be utilized as a new starting point and the process can, for example, be run again. As illustrated in FIG. 6(b), a new series of rays can thus be emitted out from an exemplary initial average point 610, and, for example, a new average point 620 can be calculated.

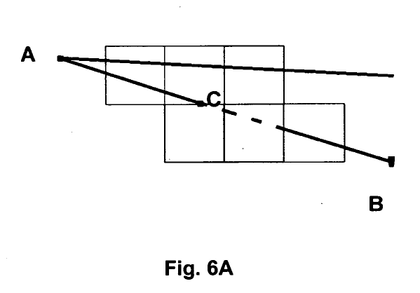

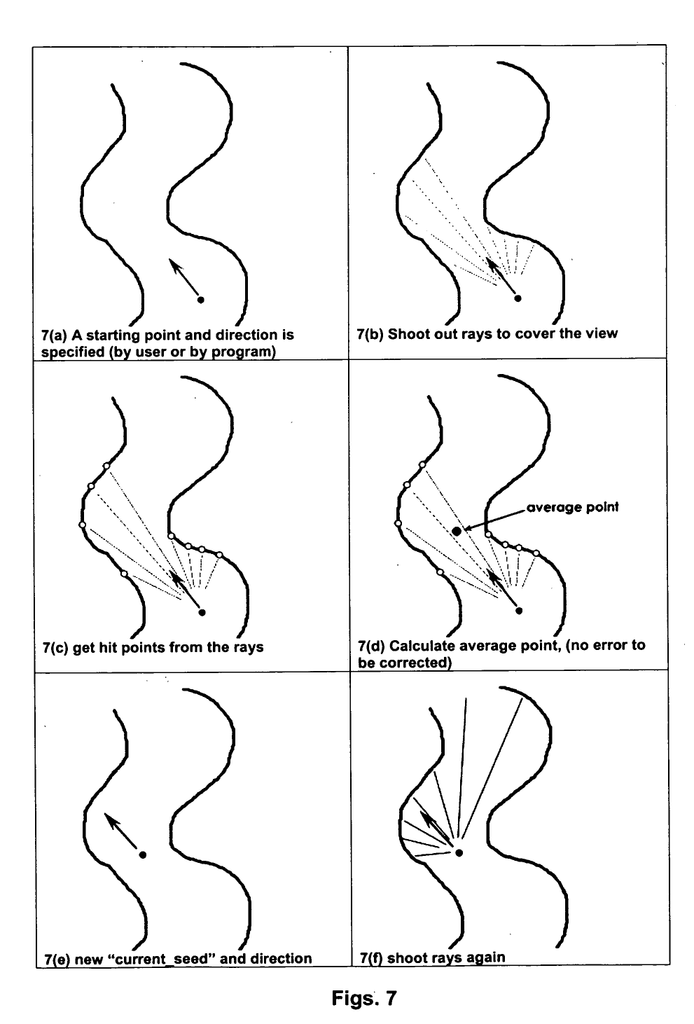

By successively executing this procedure, a series of such average points can be, for example, designated along the lumen of the tube-like structure, as illustrated in FIG. 6(c). This series of points can, for example, be used as a set of control points of a curve 630 in 3D space, which is actually a centerline describing the shape of the tube-like structure. The centerline generation process is illustrated in greater detail in FIG. 7, described below.

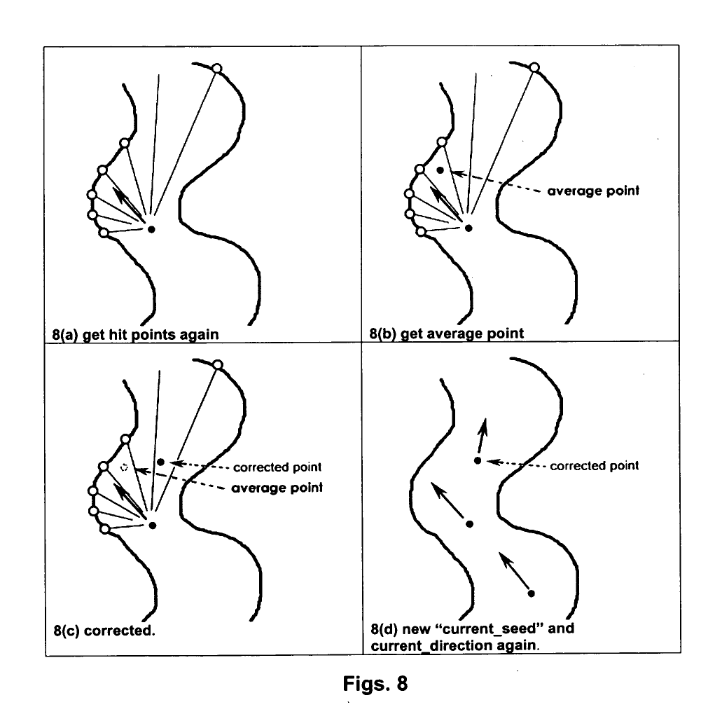

Since the above described process is an approximation of the actual geometrical “center” of the lumen, in exemplary embodiments of the present invention further checks can be implemented to ensure that the approximation is valid. For example, when each average point is found, additional rays can be shot from the average point against the surrounding wall, and the distances between the average point and the wall surface checked. If the average point is found to be too close to one side of the lumen, then it can be “pushed” towards the other side. This process is illustrated in FIGS. 8, as described below.

In exemplary embodiments of the present invention the above described ray shooting algorithm can be implemented, for example, according to the following pseudocode:

Exemplary Pseudo Code for Centerline Generation Using Ray Shooting

Function GenerateCenterline:
Input: The lumen volume,

the starting point and starting direction (by user or by program),
the end point (by user or by program)

Output: A series of points inside the lumen forming a centerline of the lumen
Function body:
{

| | |
|---|---|
| Create empty centerline_point_list; | //initialization |
| current_seed = starting point; | //initialization |
| current_direction = starting direction; | //initialization |

| | |
|---|---|
| centerline_point_list.add(current_seed); | //add the starting point |

While ( (distance between current_seed and end point) > MIN_DISTANCE)
{

hit_points = ShootRays(current_seed, current_direction, N);

//shoot N rays from current_seed, towards current_direction, spread the
//rays out in a pattern such that they cover the whole image plane;
//collect N hit points resulting from the shooting ray;

p = avrg(hit_points.x, hit_points.y, hit_points.z);

//compute the averages of x, y, z coordinates of all the N hit points;
//set a new point p = (avrg(x), avrg(y), avrg(z));

| ErrorCorrection(p); | //error correction if p happens |
|---|---|
| | //to be not at center of lumen |
| current_direction = p-current_seed; | //new direction from the |

//seed to new point

| | |
|---|---|
| current_seed = p; | //new seed point |
| centerline_point_list.add(current_seed); | //add as centerline_point |

}
}//end of function GenerateCenterline
Function ShootRays:

| Input: vol - | | The lumen volume, |
|---|---|---|
| | Start - | the ray start point, |
| | Direction - | the main direction, |
| | N - | the number of rays to shoot |

Output: The hit points
Function Body:
{

| | |
|---|---|
| InitRays(N); | //initialize the directions of the N rays to |

//cover the current image plane

For (all N rays Rₙ)

hitPointsₙ = ShootSingleRay( );

Return hitPoints;

}
Function ErrorCorrection:

//this can be done in various ways
//one way:
Shoot M Rays to all the directions perpendicular to the current_direction;
Calculate the distances between the hit points and point P;
If some of the distance is too short comparing with the average of all the distances, the point
P might be too close to one side of the lumen wall, so put it to another side.

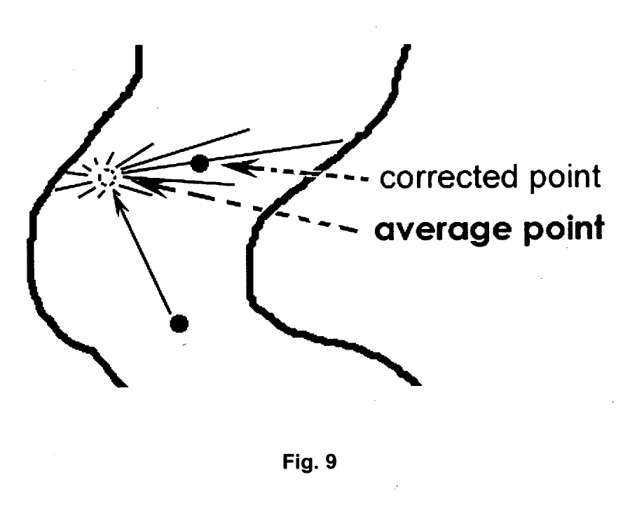

FIGS. 7(a) through 7(f) illustrate the steps in the GenerateCenterline function where no error in the position of the average point exists, and FIGS. 8(a) through 8(d) illustrate the steps in the ErrorCorrection function, where error is found in the position of an average point, of the exemplary pseudocode presented above. FIG. 9 illustrates in detail how rays are shot from an average point after it has been designated to verify if its position is correct. With reference to FIG. 9, because the initial average point was too close to the left side of the lumen wall, the corrected point is taken as the next seed point from which the next set of rays is shot.

Dynamic Stereoscopic Convergence

In exemplary embodiments of the present invention, ray shooting techniques can also be utilized to maintain optimum convergence of a stereoscopically displayed tube-like structure. In order to describe this functionality, a brief introduction to stereo convergence is next presented.

When displaying 3D objects stereoscopically, in order to give a user the correct stereographic effect as well as to emphasize the area of interest of the object being displayed, the convergence point needs to be carefully placed. This problem is more complex when producing stereoscopic endoscopic views of tube-like structures, since the convergence point's position in the 3D virtual space becomes an important factor affecting the quality of the display.

As is known in the art, the human pair of eyes are about 65 mm apart from each other on average. Thus, each eye sees the world from slightly different angles and therefore gets different images. The binocular disparity caused by this separation provides a powerful depth cue called stereopsis or stereo vision. The human brain processes the two images, and fuses them into one that is interpreted as being in 3D. The two images are known as a stereo pair. Thus the brain can use the differences between the stereo pair to get a sense of the relative depth in the combined image.

How Human Eyes Look at Objects:

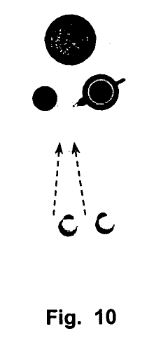

In real life when people are looking at a certain object, their two eyes are focusing on the object, which means the two eyes' respective viewing directions cross at that point. The image of that point is placed at the center of both eyes' field of view. This is the point at which people can see things clearly and most comfortably, and is known as the convergence point. At positions other than this point, objects are not the center of the eyes' field of view, or they are out of focus, so people will pay less attention to them or will not be able to see them clearly. FIG. 10 illustrates this situation. FIG. 10 is a top view of two eyes looking at the spout of a teapot. The other part of the teapot as well as the other depicted objects will not be at the center of the field of view, and are thus too near or too far to be seen clearly.

When people want to see the other parts of a scene, their eyes change to focus on another position, so as to keep the focused point (the new cross of viewing directions) on the new spot of interest.

The Camera Analogue:

Thinking of two eyes as two cameras focusing on the same point is illustrated in FIG. 11 and FIG. 12. The figures show the two cameras, their viewing direction, as well as their viewing frustum. A viewing frustum is the part of a 3D space where all the objects within can be seen by the camera and anything outside will not be seen. The viewing frusta are enclosed within the black triangles emanating form each respective camera in FIG. 11. As frusta are in 3D, in FIG. 12 they are more accurately depicted as pyramids whose vortices are at the lenses of the respective cameras.

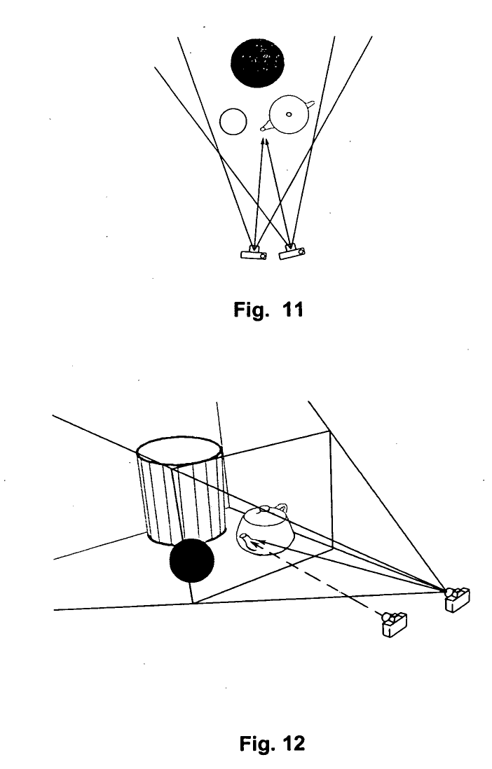

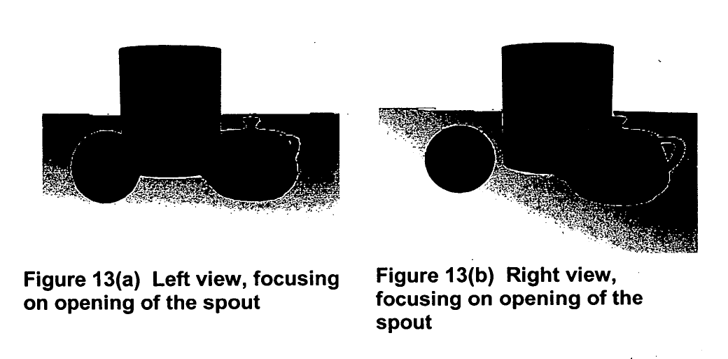

FIGS. 13(a) and (b) show exemplary images captured by each of the left and right cameras of FIGS. 11 and 12, respectively. The images obtained by the cameras are similar to those seen by two eyes, where FIG. 13(a) depicts an exemplary left eye view and FIG. 13(b) an exemplary right eye view. The images are slightly different, since they are taken from different angles. But the focused point (here the spout of the teapot) is projected at the center of both images, since the two cameras' (or two eyes') viewing directions cross at that point. When focusing on another object, the cameras will be adjusted to update to the new focus point, such that the image of the new focus point is projected at the center of the new image.

Stereo Effects in Computer Graphics:

In computer graphics applications, if, for example, stereographic techniques are used to display the two images shown in FIGS. 13(a) and 13(b) on a computer monitor, such that a user's left eye sees only the left view, and his right eye sees only the right view, such a user could, for example, be able to have depth perception of the objects. Thus, a stereo effect can be created.

In order to render each of the two images correctly however, the program needs to construct each camera's frustum, and locate the frustum at the correct position and direction. As the cameras simulate the two eyes, the shape of the frustum is the same, but the position and direction of the frusta differ as do the position and direction of two eyes.

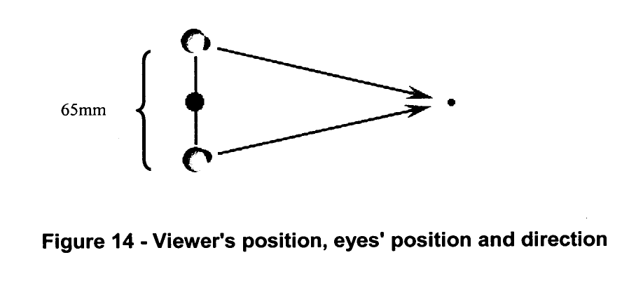

Usually the physical dimensions of a human being is not important to this process, so, for example, a viewer's current position can be approximated as a single point, and a viewer's two eyes can be placed on two sides of the viewer's current position. Since for a normal human being the two eyes are separated at about 65 mm away from each other, an exemplary computer graphics program needs to space the two frusta by 65 mm. This is illustrated in FIG. 14, where the large dot between the eyes is a user's viewpoint relative to a viewed convergence point, and the fruta are spaced 65 mm apart, with the viewpoint in their center.

After placing the two eyes' positions correctly, an exemplary program needs to set the correct convergence point, which is where the two eyes' viewing direction cross, thus setting the directions of the two eyes.

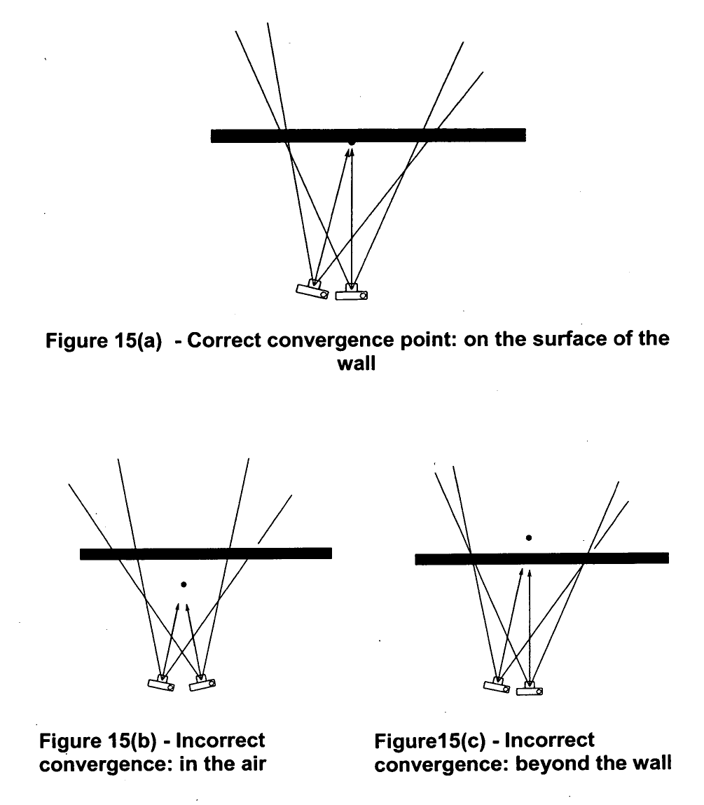

The position where the two viewing directions cross is known as the convergence point in the art of stereo graphics. In stereo display in computer graphics applications, the image of the convergence point can be projected at the same screen position for the left and right views, so that the viewer will be able to inspect that point in detail and in a natural and comfortable way. In real life the human brain will always adjust the two eyes to do this; in the above described case of two cameras the photographer takes care to do this. In computer graphics applications, a program must calculate the correct position of the convergence point and correctly project it onto the display screen. Generally, people's eyes do not cross in the air in front of an object, nor will they cross inside the object's surface. In real life when people walk inside a room or a tunnel (empty room or tunnel, without any objects inside to consider), people will naturally focusing on the walls or surfaces (there are some bumps, drawings, etc), which means, the two eyes will converge on one spot on the area of interest on the surface. Thus, in virtual endoscopy, to best simulate an actual endoscopy, a user should be guided to look at the surface of the virtual lumen. Thus, the user's eyes should not be led to cross in the air in front of the surface, or beyond the surface into the lumen wall. In order to do this, a given exemplary virtual endoscopy implementation needs to determine the correct position of the convergence point such that it is always on the surface of the area of interest of the lumen being inspected. This is illustrated in FIGS. 15(a) through (c), respectively using the cameras described above focusing on a point in 3D space.

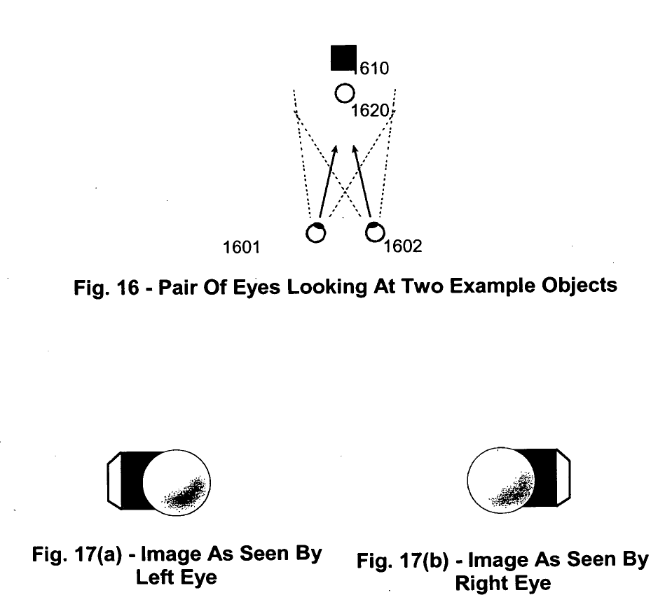

Similarly, FIG. 16 depicts a pair of eyes (1601, 1602) looking at an exemplary ball 1620 in front of an exemplary cube 1610. As noted, because human eyes are separated form each other by a few inches, the left and right eyes each see slightly different views of these objects, as illustrated in FIGS. 17(a) and (b), respectively. The dotted lines in FIG. 16 are the edges of the frustum for each eye. Thus, FIGS. 17(a) and (b) depict exemplary Left and Right views of the scene of FIG. 16, respectively. As noted, when human eyes are focused on a certain point of interest (such as, for example, the highlighted spot on the ball's surface in FIGS. 17(a) and (b)), their respective lines of sight cross at that point, i.e., the convergence point.

In stereoscopic displays on a computer screen, images such as those depicted in FIGS. 17(a) and (b) can be displayed on the same area of the screen. In exemplary embodiments of the present invention, for example, a stereoscopic view can be achieved when a user wears stereographic glasses. In other exemplary embodiments, a stereoscopic view may be achieved from a LCD monitor using a parallax barrier by projecting separate images for each of the right eye and left eye, respectively, on the screen for 3D display. In still other exemplary embodiments a stereoscopic view can be implemented via an autostereoscopic monitor such as are now available, for example, from Siemens. In still other exemplary embodiments, a stereoscopic view may be produced from two high resolution displays or from a dual projection system. Alternatively, a stereoscopic viewing panel and polarized viewing glasses may be used. The convergence point can be set to the same place on the screen, for example, the center, and a viewer can be, for example, thus guided to focus on this spot. The other objects in the scene, if they are nearer to, or further from, the user than the convergence point, can thus appear at various relative depths.

For stereoscopic display of an endoscopic view of a tube-like structure, it is important to make sure that the convergence point is correctly calculated and therefore that the stereographic images are correctly displayed on the screen, so that a user can be guided to areas that need to be paid attention to, and that distracting objects can, for example, be avoided.

In exemplary embodiments of the present invention it can be assumed, for example, that the center of the image is the most important part and that a user will always be focused on that point Oust as it is a fair assumption that a driver will generally look straight forward while driving). Thus, in exemplary embodiments of the present invention the area of the display directly in front of the user in the center of the screen can be presented as the point of stereo convergence. In other exemplary embodiments of the present invention, the convergence point can be varied as necessary, and can be, for example, dynamically set where a user is conceivably focusing his view, such as, for example, at a “hit point” where a direction vector indicating the user's viewpoint intersects—or “hits”—the inner lumen wall. This exemplary functionality is next described.

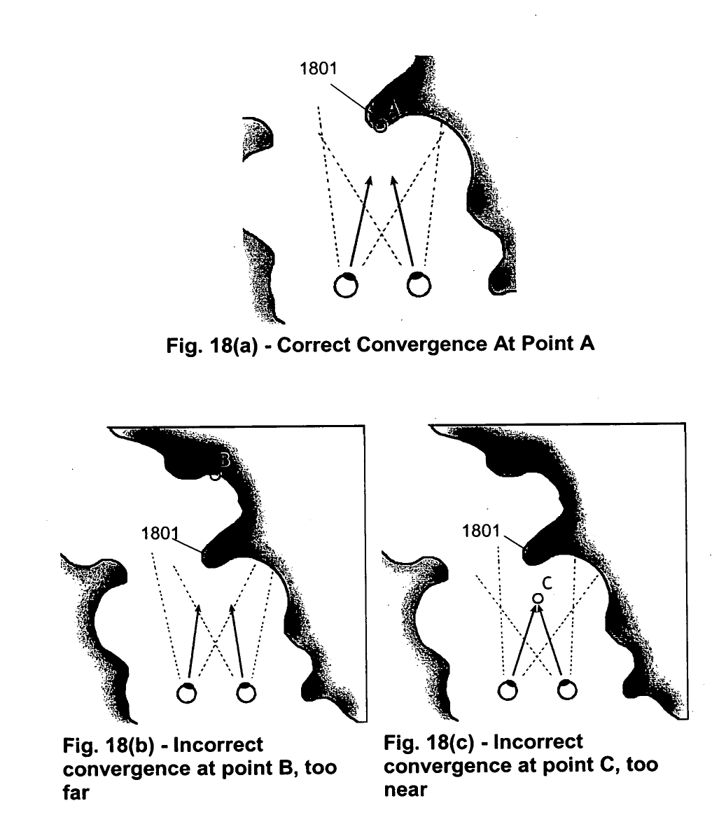

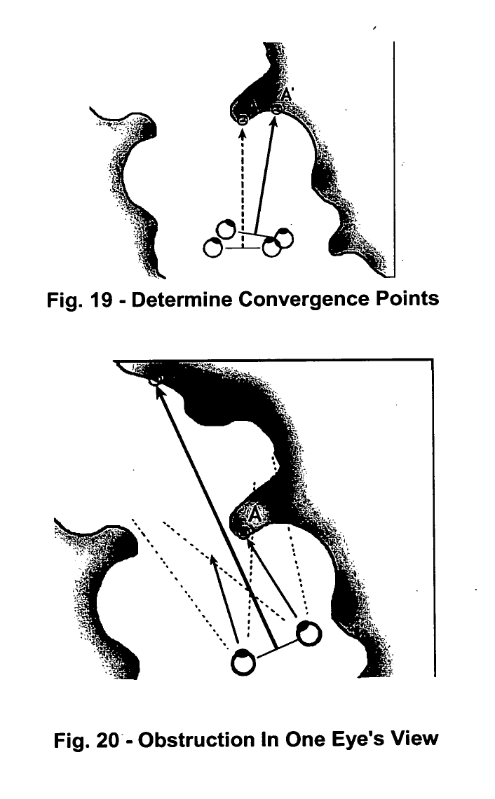

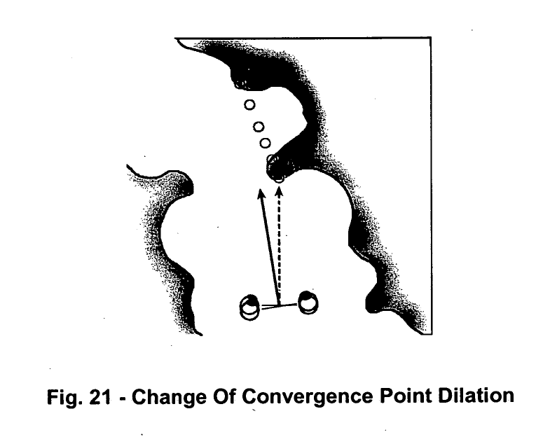

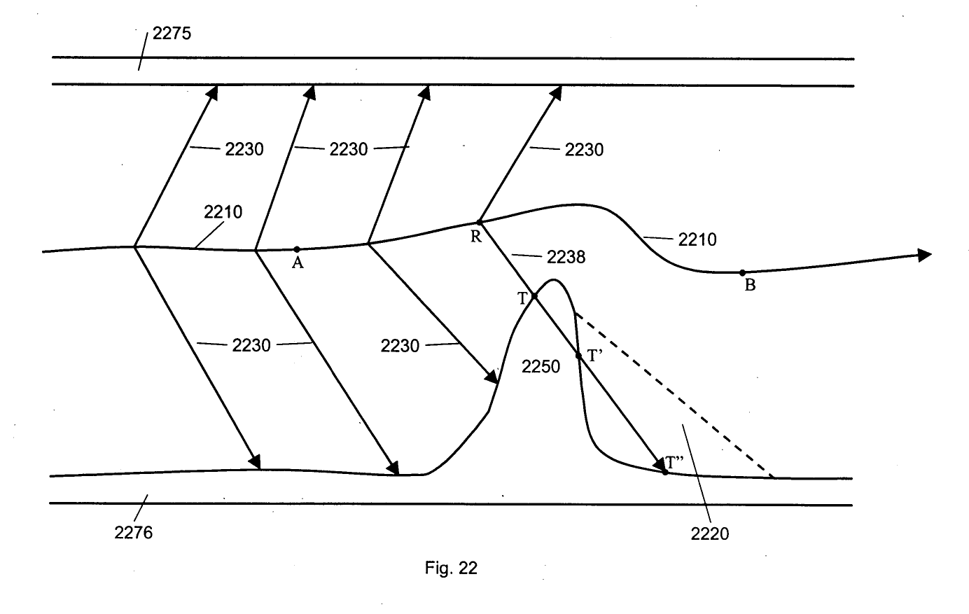

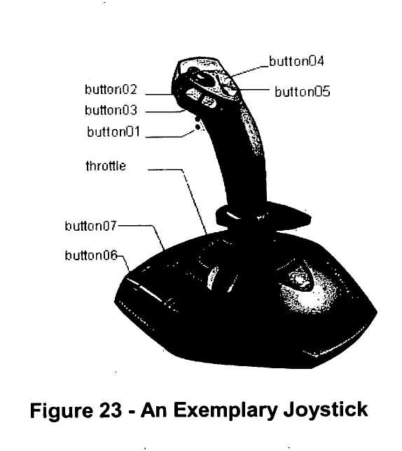

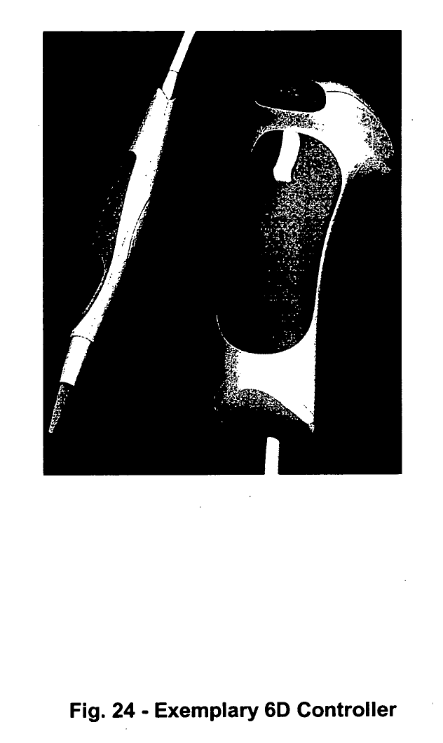

FIGS. 18 depict an exemplary inner lumen of a tube-like structure, where certain convergence point issues can arise. For a structure similar to the local region 1801 in FIG. 18(a), a user's region of interest can, for example, be near point A. The virtual endoscopy system can, for example, thus calculate and place the convergence point at point A. The same shaded region is shown, in lesser magnification, in each of FIGS. 18(b) and 18(c), also as 1801. Incorrect convergence points, as shown in FIGS. 18(a) (too far) and 18(b) (too near), can give a user distractive and uncomfortable views when trying to inspect region 1801. Thus it is key to correctly calculate and present the stereoscopic convergence point to optimize a user's viewing experience.

In exemplary embodiments of the present invention, several methods can be used to ensure a correct calculation of a stereoscopic convergence point throughout viewing a tube-like anatomical structure. Such methods can, for example, be combined together to get a very precise position of the convergence point, or portions of them can be used to get good results with less complexity in implementation and computation.

The shooting ray technique described above can also be used in exemplary embodiments of the present invention to dynamically adjust the convergence point of left eye and right eye views, such that a stereo convergence point of the left eye and right eye views is always at the surface of the tube-like organ along the direction of the user's viewpoint from the center of view. As noted above, stereo display of a virtual tube-like organ can provide substantial benefits in terms of depth perception. As is known in the art of stereoscopic display, stereoscopic display assumes a certain convergence distance from a user viewpoint. This is the point the eyes are assumed to be looking at. At that distance the left and right eye images have most comforatable convergence. If this distance is kept fixed, as a user moves through a volume looking at objects which may have distances from this viewpoint which can vary from the convergence distance, it can place some strain on the eyes to continually adjust. Thus, it is desirable to dynamically adjust the convergence point of the stereo images to be at or near the object a user is currently inspecting. This point can be automatically acquired by shooting a ray from the viewpoint (i.e., the center of the left eye and right eye positions used in the stereo display) to the colon wall along a direction perpendicular to the line connecting the left eye and right eye viewpoints. Thus, in exemplary embodiments of the present invention, when the eyes change to a new position due to a user's movement though the tube-like structure, the system can, for example, shoot out a ray from the mid point between the two eyes towards the viewing direction.

For ray shooting, when the eye separation is not significant compare with the distance from the user to the wall in front of the user, it can be, in exemplary embodiments of the present invention, assumed that the two eyes are at the same position, or, equivalently that there is only one eye. Thus, most of the calculations can be, for example, done using this assumption. In the case the difference between the two eyes is important, the two eyes should be considered individually, rays might be shot out from two eyes' position individually. The ray may pick up the first point that is opaque along its path. This point may be the surface that is in front of the eyes and is the point of interest. The system can, for example, then use this point as the convergence point to render the images for the display.

FIG. 19 illustrates a method of determining convergence points according to an exemplary embodiment of the present invention. In one instance, the ray shoots out from the mid point between the eyes, and picks up point A. The system may set A as the convergence point for the rendering process. At the next instance, when the eyes have moved slightly to the right, another ray shoots out and picks up point A′ as the convergence point for an updated rendering. Thus, the user's convergence point may always be directed towards the point of interest of the subject. This exemplary method works effectively in most instances.

In exemplary embodiments of the present invention the above described ray shooting algorithm can be implemented, for example, according to the following pseudocode:

For every display loop,

shoot ray to get a hit point;
if get a hit point, set it as the convergence point.

Thus:
Input: user's position, viewing direction, volume
Output: new convergence point
Function UpdateConvergencePoint:
{

create ray from the user's position along the viewing direction;
hitPoint = shootSingleRay(ray);
distance = CalculateDistanceFromUserPosition(hitPoint);
if(distance > MIN_CONVERGENCE_DISTANCE) set as
new convergence point;

}

It is noted that this method may fail, when the eye separation is significant in relation to the distance between a user and the lumen wall in front of the user. As is illustrated in FIG. 20, the convergence point determined using the above described method should be A′, as this is the nearest hit point along the direction of the viewpoint, indicated by the long vector between the viewpoint and point A′. While this convergence point would be correct for the left eye, which can see point A′, for the right eye the convergence point should actually be point A, because, due to the protrusion of a portion of the lumen wall, the right eye cannot see point A′, but sees point A. If the convergence point is thus set at A′, a user would see an unclear obstruction with his right eye, which can be distractive and uncomfortable.

Accordingly, in exemplary embodiments of the present invention, after determination of the convergence point using the method described above, an exemplary system can, for example, double check a result by shooting out two rays, one from each of the left and right eyes, which can then, for example, obtain two surface “hit” points. If the system finds the convergence point found with the above described method to be identical with the new points, that confirms the convergence point's viability. This is the situation in FIGS. 18(a) and 19, where both eyes converge at the same point, A and A′, respectively. If, however, the situation depicted in FIG. 20 occurs, then there will be a conflict and the actual convergence point should not be the hit point along the viewpoint direction A′. If this situation is detected the user may be too close to the lumen wall and thus running into obstructions of his view. If the user cannot approach the lumen wall too closely, this problem can be avoided. Alternatively, the convergence point can be set at some compromise point, and while both point A and point A′ will be slightly out of convergence, it may be acceptable for a short time. A user can, in exemplary embodiments of the present invention, in such instances be advised via a pop-up or other prompt that at the current viewpoint stereo convergence cannot be achieved for both eyes.

Thus, in exemplary embodiments of the present invention, by collecting information regarding hit points as depicted in FIGS. 7, an exemplary system can use the distances from a user's viewpoint to the surrounding walls to detect any possible “collision” and prevent a user from going into the wall for example, by displaying a warning pop-up or other informative prompt.

As the viewer moves inside the tube-like structure, the convergence point may change back and forth rapidly. This may be distracting or uncomfortable for a user. In an exemplary embodiment of the invention, the convergence points in consecutive time frames can be, for example, stored and tracked. If there is a rapid change, an exemplary system can purposely slow down the change by inserting a few transition stereo convergence points in between. For example, as illustrated in FIG. 21, the convergence point needs to be changed from point A to A′ as a user turns the viewpoint to the left (counterclockwise), but the exemplary system inserts a few interpolated convergence points in between points A and A′ so as to give a user the visual effect of a smoother transition as opposed to immediately “jumping” from A to A′, which will generally be noticeable.

Rendering Folds Transparently to View Occluded Voxels Behind Them

In exemplary embodiments according to the present invention, a ray shooting technique, as described above in connection with maintaining proper stereoscopic convergence and centerline generation, can be similarly adapted to the identification of “blind spots.” This technique, in exemplary embodiments of the present invention, can be illustrated with reference to FIG. 22. FIG. 22 depicts a longitudinal cross-section of a colon lumen. Visible are the upper colon wall 2275 and the lower colon wall 2276. Also visible is a centerline 2210, which can be calculated according to the ray shooting technique described above or using other techniques as may be known in the art. Finally, there is visible a protrusion 2250 from the bottom colon wall. Such protrusion can be, for example, a fold in the colon wall or it can be, as depicted in FIG. 22, for example, a polyp. In either event, the diameter of the colon lumen is decreased near such protrusions. Thus, the centerline 2210 must move upward above polyp 2250 to adjust for this decreased diameter. In the example schematic of FIG. 22, it is assumed that a user is virtually viewing the colon moving from the left of the figure to the right of the figure in a fly-through or endoscopic view. Thus, while a user moves through the colon in the direction from left to right (as indicated by the arrow at the end of centerline 2210), voxels associated with the colon areas behind a protrusion such as polyp 2250 will not be visible to a user from a viewpoint moving along center line 2210.

Conventionally, users of virtual colonoscopies “fly-through” a colon and keep their viewpoint pointed along the centerline in the forward direction, or following centerline 2210 with reference to FIG. 22. If they cannot see voxels which are forward of (and thus “behind”) a protrusion such as polyp 2250 or a fold in the colon wall they must first pass the protrusion, then stop and change their viewpoint to point downwards or upwards, as the case may be, and look behind the protrusion. With reference to FIG. 22 this could be effected from viewpoint B. A user noticing polyp 2250 at point A could see that there was a blind spot 2220 behind the polyp as a result of its protrusion into the colon lumen. The only way to inspect the voxels 2220 in the “blind spot” of the polyp 2250 would be to stop at a viewpoint such as, for example, B, and change the viewpoint to look downward at the area of blind spot 2220. This is tedious, and requires more user interaction than simply watching the fly-through view on the screen. Thus, it is disfavored by users, such as for example, radiologists. Accordingly, in exemplary embodiments according to the present invention, a ray shooting technique can be used to locate blind spots such as, for example, blind spot 2220. Once located, in exemplary embodiments of the present invention the protrusions can be rendered as transparent as a user's viewpoint comes close to the protrusions such as, for example, at point A in FIG. 22.

Shown in FIG. 22 are a variety of rays 2230 and one special ray is 2238. Rays 2230 can be, for example, shot out from the centerline to the colon wall inner surface. Because there is a change in voxel intensity between the inner colon lumen (which is generally full of air) and the inner colon lumen wall it is easy to detect when a ray has hit a wall voxel, as described above in connection with centerline generation and stereoscopic convergence points. If two rays 2230 are each shot out from centerline 2210 at approximately equal angles ot the centerline direction, by virtue of orignating on the centerline the distances to the inner colon wall should be within a certain percentage of each other. However, if there is a protrusion on one side of the colon wall but not on the other, such as is the case near the polyp 2250 where the upper ray sent from point R 2230 hits the colon wall but the lower ray 2238 hits a colon lumen/wall interface at the top of polyp 2250 at point T, continues through the polyp to point T′ and hits a third wall/air interface at T″, it can, in exemplary embodiments of the present invention, be detected that there is a protrusion and therefore a blind spot.

In alternate embodiments of the present invention, other algorithms can use not just how many times a ray has crossed a lumen/lumen wall interface but can determine that a protrusion is occurring due to significantly shorter distances acquired between rays 2230 and 2238 when shot from appropriate points upstream from (i.e., prior to reaching, or to the left of point R in FIG. 22) the protrusion. Once having detected a protrusion or polyp in the colon lumen, and therefore a blind spot, in exemplary embodiments of the present invention various functionalities can be implemented. A system can, for example, alert a user that a blind spot is approaching and can, for example, prompt the user to enter a “display protrusion as transparent” command, or a system can, for example, slow down the speed with which the user is moved through the colon lumen such that the user has enough time to first view the protrusion after which the protrusion can morph to being transparent, thus allowing the user to see the voxels and the blind spots without having to change his viewpoint as he moves through the colon.

In exemplary embodiments according to the present invention, blind spots can be, for example, detected as follows. While a user takes, for example, a short (2-5 minute) break, an exemplary system can generate a polygonized surface of an inner colon wall, resulting in the knowledge of the spatial position of each polygon. Alternatively, a map of all voxels along the air/colon wall interface could be generated, thus identifying their position. Then an exemplary system can, for example, simulate a fly-through along the colon lumen centerline from anus to cecum, and while flying shoot rays. Thus the intersection between all of such rays and the inner colon wall can be detected. Such rays would need to be shot in significant numbers, hitting the wall at a density of, for example, 1 ray per 4 mm². Using this procedure, for example, a map of the visible colon surface can be generated during an automatic flight along the centerline. The visible surface can then be subtracted from the previously generated surface of the entire colon wall, with the resultant difference being the blind spots. Such spots can then be, for example, colored and patched over the colon wall during the flight or they can be used to predict when and to what extent to render certain parts transparent.

In alternate exemplary embodiments of the present invention, another option to view a blind spot is to fly automatically along the centerline towards it, stop, and then turn the view towards the blind spot. This would not require setting any polyps to be transparent. This could be achieved, for example, by determining the closest distance of all points within or along the circumference of a given blind spot to the centerline and then determine an average point along the centerline from which all points on the blind spot can be viewed. Once the journey along the centerline has reached this point, the view can be, for example, automatically turned to the blind spot. If the blind spot is too big to be viewed in one shot, then, for example, the fly-over view could be automatically adapted accordingly or, for example, the viewpoint could move until the blind spot is entirely viewed, all such automated actions being based upon ray-shooting using feedback loops.

In exemplary embodiments of the present invention the blind spot detection process can be done a priori, at a pre-processing stage, as described above, such that the system knows before the user arrives there where the blind spots are, or in alternative embodiments according to the present invention, it can be done dynamically in real time, and when a user reaches a protrusion and a blind spot a system can, for example, (i) prompt the user for transparency commands, as described above, (ii) change the speed with which the user is brought through the colon and automatically display the protrusion transparently after a certain time interval, or (iii) take such other steps as may be desirable.

Interactive Display Control Interface

As noted above, due to the historico-cultural fact that virtual viewing of three-dimensional data sets was first implemented on standard PC's and similar devices, conventional systems for navigating through a three-dimensional volume of a tube-like structure, such as the colon, generally utilize a mouse (or other similar device, e.g., a track ball) as the sole user control interface. Inasmuch as a mouse or other two-dimensional device is in fact designed for navigating in two dimensions within the confines of a document, image or spread sheet, using a mouse is sometimes a poor choice for navigating in three-dimensions where, in fact, there are six degrees of freedom (translation and rotation) as opposed to two.

In general, a conventional two-button or wheel mouse has only two buttons or two buttons and one wheel, as the case may be, to control all of the various movements and interactive display parameters associated with virtually viewing a tube-like anatomical structure such as, for example, a colon. The navigation through three-dimensional volume renderings of colons, blood vessels and the like in actuality require many more actions than three. In order to solve this problem, in an exemplary embodiment according to the present invention directed to virtual viewing of the colon, a gaming-type joystick can be configured to provide the control operations as described in Table A below. It is noted that a typical joystick allows for movement in the X, Y, and Z directions and also has numerous buttons, both on its top and its base, allowing for numerous interactive display parameters to be controlled. FIG. 23 depicts such an exemplary joystick.

TABLE A
Exemplary Mapping of Control Functions onto a Joystick

| Joy Stick | | |
|---|---|---|
| Button | Handle | Effect/function |

Navigation:

| | | |
|---|---|---|
| Button02 | | Toggle guided moving toward cecum |
| Button03 | | Toggle guided moving toward rectum |
| | | Toggle guided/manual mode |
| | | Toggle view toward cecum/rectum |
| Button04 | | Change view toward cecum |
| Button05 | | Change view toward rectum |
| Button02 (when | | Toggle manual forward |
| manual mode) | | |
| Button03 (when | | Toggle manual backward |
| manual mode) | | |
| Rotate(Look Around) | | |
| NONE | Left | Yaw |
| | Right | Yaw |
| | Front | Pitch |
| | Back | Pitch |
| Twist CCW | | Roll CCW |
| Twist CW | | Roll CW |
| Zoom | | |
| Button01 (trigger) | | Zoom up the 3-side view with the |
| | | targeted point as the center |
| Place Marker | | |
| Button06 | | Set starting point |
| Button06 again | | Set ending point to complete the marker |
| Button07 | | Remove the last completed/uncompleted |
| | | marker. |

With reference to Table A above, the following interactive virtual viewing operations can be enabled in exemplary embodiments of the present invention.

A. Navigation

In an exemplary embodiment of the present invention, navigation through a virtual colon can be controlled by the use of four buttons on the top of the joystick. Such buttons are normally controlled by the thumb of the user's hand, which the user uses to operate the joystick. For example, Button02, appearing at the top left of the joystick, can toggle between guided moving toward the cecum and manual moving toward the cecum. Button03 is used for toggling between guided and manual moving toward the rectum, or backward in the standard virtual colonoscopy. It is noted that in the standard virtual colonoscopy a user navigates from the rectum toward the cecum, and that is known as the “forward” direction. Thus, in exemplary embodiments of the present invention, it is convenient to assign one button to toggle between manual and guided moving towards the cecum and having another button assigned to toggle between guided and manual motion towards the rectum, whether those directions are nominally assigned the terms “forward” or “backward” will depend upon the application. Notwithstanding whether the direction through the virtual colon is towards the rectum or towards the cecum, a user is free to choose whether the view is towards the rectum or towards the cecum. Thus, there are four possibilities: moving towards the cecum, viewing “backwards” or towards the rectum, moving towards the rectum and viewing towards the rectum, or moving towards the rectum and viewing towards the cecum. Therefore, in exemplary embodiments according to the present invention Button 04 can be used to change the view towards the cecum and Button 05 can be used to change the view towards the rectum.

B. Rotation (Looking Around)

As is known, in a three-dimensional data set or, in general in any motion in three-dimensions, one can rotate about either the X, Y or Z axis in viewing anatomical tube-like structures in a virtual three-dimensional volumetric rendering. It is often convenient to use rotation to “look around” the area where the user's virtual point of view is. Thus, since rotation can be either clockwise or counterclockwise or right handed or left handed with respect to a particular axis, there are six degrees of rotational freedom. In exemplary embodiments according to the present invention, as noted in Table A these six degrees of rotational freedom can be implemented using six control actions. Moving the joystick left or right controls yaw in either of those directions, moving the joystick front or back controls pitch in either of those directions, and twisting the joystick clockwise or counterclockwise will effect a roll clockwise or counterclockwise. It is noted that twisting the joystick clockwise or counterclockwise is about or with respect to the positive Z axis of the joystick which comes up through and points upward therefrom.

C. Zoom/Zoom Up Three-Sided View

In many virtual colonoscopy implementations it is highly useful, and arguably necessary, to have some kind of zoom functionality whereby the user can expand the scale of the voxels that he views with respect to display. This is, in effect, a digital magnification of a particular set of voxels within the three-dimensional data set. In exemplary embodiments of the present invention, implementing interactive display controls with the joystick, a trigger button can be used to implement zoom whenever a user moving through a colon desires to magnify a portion of it, and simply pulls on the trigger and the zoom is implemented with the targeted point as the center.

Alternatively, a trigger or other button could be programmed to change the cross sectional point for the display of axial, coronal and saggital images. For example, if no trigger or other so assigned button is pressed, the cross-sectional point for the display of axial, coronal and saggital images can be oriented at the online position of a user. If such trigger or other button is pushed, the cross-sectional point can, for example, become the point on the tube-like organ's interior wall where a virtual ray shot from the viewpoint hits. This can be used to examine wall properties at a given point, such as at a suspected polyp. At such point the axial, coronal and saggital images can be displayed in a digitally magnified mode, such as, for example, 1 CT pixel mapped to two monitor pixels, or any desired zoom mapping.

D. Place Marking

In virtual colonoscopies and endoscopies it is often convenient to be able to set a starting point and an ending point to be viewed on a particular pass through a portion of the colon. The user can set a starting point in exemplary embodiments according to the present invention by pressing Button06 and can set an ending point by pressing Button06 again to complete the marker. In exemplary embodiments according to the present invention, Button06 is located on the base of a joystick, inasmuch as it is not used continually through the virtual viewing as are the other functionalities whose control has been implemented using buttons on the joystick itself. If a user should desire to remove the last completed or uncompleted marker set using Button06, in exemplary embodiments of the present invention she can push Button07 also located, in exemplary embodiments according to the present invention, on the base of the joystick.

In alternative exemplary embodiments according to the present invention, control functions can be mapped to a six degree of freedom (6D) controller, an example of which is depicted in FIG. 24 (on the right, a stylus is shown on the left). An exemplary 6D controller consists of a six degree of freedom tracker with one or more buttons. The trackers can, for example, use radio frequencies, or can, for example, be optical trackers, or use some other technique as may be known in the art. Buttons mounted on the device enable a user to send on/off signals to the computer. By combining the buttons and 6D information from these devices, one can map user commands to movements and activities to be performed during exploration of a tube-like structure. For example, a user could be shown on the screen a virtual representation of the tool (not a geometrical model of the device, but a symbolic one) so that moving and rotating the device shows exactly how the computer is interpreting the movement or rotation.

It is noted that a 6D controller can provide more degrees of freedom and can thus allow greater flexibility in the mapping of actions to commands. Further, such a control interface involves less mechanical parts (in one exemplary embodiment just a tracker and a button) so that it is less likely to break down due to usage. Since there is no physical contact between a user and the tracking technology (generally RF or optical) it can be more robust.

Exemplary Systems

The present invention can be implemented in software run on on a data processor, in hardware in one or more dedicated chips, or in any combination of the above. Exemplary systems can include, for example, a stereoscopic display, a data processor, one or more interfaces to which are mapped interactive display control commands and functionalities, one or more memories or storage devices, and graphics processors and associated systems. For example, the Dextroscope and Dextrobeam systems manufactured by Volume Interactions Pte Ltd of Singapore, runing the RadioDexter software, or any similar or functionally equivalent 3D data set interactive display systems, are systems on which the methods of the present invention can easily be implemented.

Exemplary embodiments of the present invention can be implemented as a modular software program of instructions which may be executed by an appropriate data processor, as is or may be known in the art, to implement a preferred exemplary embodiment of the present invention. The exemplary software program may be stored, for example, on a hard drive, flash memory, memory stick, optical storage medium, or other data storage devices as are known or may be known in the art. When such a program is accessed by the CPU of an appropriate data processor and run, it can perform, in exemplary embodiments of the present invention, methods as described above of displaying a 3D computer model or models of a tube-like structure in a 3D data display system.

The present invention has been described in connection with exemplary embodiments and implementations, as examples only. It is understood by those having ordinary skill in the pertinent arts that modifications to any of the exemplary embodiments or implementations can be easily made without materially departing from the scope or spirit of the present invention, which is defined by the appended claims.

1. A method of virtually displaying a tube-like anatomical structure, comprising:
obtaining scan data of an area of interest of a body which contains a tube-like structure;
constructing a volumetric data set from the scan data;
virtually displaying some or all of the tube-like structure by processing the volumetric data set,
wherein the tube-like structure is displayed stereoscopically.

2. The method of claim 1, wherein a small segment of the tube-like structure is displayed in a main viewing window, and the inner wall of the entire tube-like structure is displayed transparently in an adjacent overall view window.

3. The method of claim 2, wherein the overall view window has additional visual aids including one of path traversed so far and current position within tube-like structure.

4. The method of claim 1, wherein the tube-like structure can be displayed using a variety of stereoscopic formats, including anaglyphic red-green stereo, anaglyphic red- blue stereo, anaglyphic red-cyan stereo, interlaced display and autostereoscopic display.

5. The method of claim 1, wherein a small segment of the tube-like structure is displayed at any given time in a fly-through interactive display.

6. The method of claim 1, wherein the wall of the tube-like structure is displayed using a variety of color lookup tables.

7. The method of claim 1, wherein the wall of the tube like structure is extracted from the volumetric data set based upon a difference in voxel intensity between the tube-like structure and the air within it.

8. The method of claim 1, wherein the tube-like structure is a human or mammalian colon.

9. The method of claim 1, wherein the tube-like structure is a human or mammalian artery or vascular structure.

10. A method of generating a centerline of a tube-like structure, comprising:
shooting a set of rays from a first viewpoint;
obtaining a set of points on the inner wall of the structure where the rays hit;
averaging the three-dimensional co-ordinates of the hit points to obtain a centerline point;
using the centerline point as the next viewpoint;
repeating the process until the end of the tube-like structure has been reached; and
connecting all of the centerline points.

11. The method of claim 10, wherein the tube-like structure is a colon and wherein the first viewpoint is at or near either the rectum or the cecum.

12. The method of claim 10, wherein after obtaining each centerline point, an additional set of rays are shot from it to verify its validity as a centerline point.

13. The method of claim 12, wherein the additional set of rays are shot from the tentative centerline point in directions perpendicular to the then current viewing direction.

14. The method of claim 13, wherein if as a result of the additional ray shooting the tentative centerline point is found not to be at a position equidistant from the colon wall the centerline point is moved to a corrected position.

15. A method of dynamically adjusting a stereoscopic convergence point for viewing a tube-like structure, comprising:
shooting a ray from a viewpoint along the direction of the viewpoint;
obtaining a point on the inner wall of the structure where the ray hits;
setting the hit point as the stereoscopic convergence point.

16. The method of claim 15, further comprising testing the stereoscopic convergence point by shooting additional rays from each eyepoint and analyzing their hit points.

17. The method of claim 15 wherein the process is repeated each time the viewpoint changes.

18. The method of claim 17, wherein if the co-ordinates of the stereoscopic convergence point change from one to the next in excess of a predetermined amount, one or more intermediate stereoscopic convergence points are interpolated between the prior stereoscopic convergence point and the next stereoscopic convergence point.

19. A method of optimizing user interaction with and control of a display of a tube-like organ obtained by volume rendering of a three-dimensional data set, comprising:
mapping navigation and control functions to one or more of a joystick and a 6D controller.

20. The method of claim 19, wherein the tube-like organ is a human colon, and the mapped functions include one or more of translation in each of three dimensions, yaw, pitch, clockwise roll, counterclockwise roll, guided moving toward cecum, guided moving towards rectum, manual moving towards, cecum, manual moving towards rectum, viewpoint direction, set starting point, set ending point and zoom.

21. A method of interactively virtually displaying a tube-like structure, comprising:
obtaining scan data of an area of interest of a body which contains a tube-like structure;
constructing a volumetric data set from the scan data;
virtually displaying some or all of the tube-like structure by processing the volumetric data set;
displaying the tube-like structure stereoscopically; and
using ray shooting techniques to:
calculate a centerline of the tube-like structure; and
dynamically adjust a stereo convergence point of a viewpoint as that viewpoint is moved within the tube-like structure.

22. The method of claim 21, wherein the viewpoint is automatically moved within the tube-like structure.

23. The method of claim 21, wherein the viewpoint is moved within the tube-like structure by the interactive control of a user.

24. The method of claim 21, wherein ray shooting techniques are additionally used to warn a user when the viewpoint is within a predetermined distance of an obstacle.

25. The method of claim 24, wherein ray shooting techniques are additionally used to detect one or more of folds in a wall of the tube-like structure and blind spots behind said folds.

26. The method of claim 25, wherein when the fold is detected it is set to be transparent when the viewpoint is within a predetermined distance of the fold.

27. A computer program product comprising a computer usable medium having computer readable program code means embodied therein, the computer readable program code means in said computer program product comprising means for causing a computer to:
obtain scan data of an area of interest of a body which contains a tube-like structure;
construct a volumetric data set from the scan data; and
virtually display some or all of the tube-like structure by processing the volumetric data set,
wherein the tube-like structure is displayed stereoscopically.

28. A program storage device readable by a machine, tangibly embodying a program of instructions executable by the machine to perform a method for virtually displaying a tube-like anatomical structure, said method comprising:
obtaining scan data of an area of interest of a body which contains a tube- like structure;
constructing a volumetric data set from the scan data;
virtually displaying some or all of the tube-like structure by processing the volumetric data set,
wherein the tube-like structure is displayed stereoscopically.

29. A computer program product comprising a computer usable medium having computer readable program code means embodied therein, the computer readable program code means in said computer program product comprising means for causing a computer to:
obtain scan data of an area of interest of a body which contains a tube-like structure;
construct a volumetric data set from the scan data;
virtually display some or all of the tube-like structure by processing the volumetric data set;
display the tube-like structure stereoscopically; and
use ray shooting techniques to:
calculate a centerline of the tube-like structure; and
dynamically adjust a stereo convergence point of a viewpoint as that viewpoint is moved within the tube-like structure.

30. A program storage device readable by a machine, tangibly embodying a program of instructions executable by the machine to perform a method for virtually displaying a tube-like anatomical structure, said method comprising:
obtaining scan data of an area of interest of a body which contains a tube-like structure;
constructing a volumetric data set from the scan data;
virtually displaying some or all of the tube-like structure by processing the volumetric data set;
displaying the tube-like structure stereoscopically; and
using ray shooting techniques to:
calculate a centerline of the tube-like structure; and
dynamically adjust a stereo convergence point of a viewpoint as that viewpoint is moved within the tube-like structure.
